(19) United States
(12) Patent Application Publication
(10) Pub. No.: US 2022/0304584 A1
(43) Pub. Date: Sep. 29, 2022

(54) SYSTEM FOR USING RADIOFREQUENCY AND LIGHT TO DETERMINE PULSE WAVE VELOCITY

(71) Applicant: ZOLL Medical Israel Ltd., Kfar-Saba (IL)

(72) Inventors: Uriel Weinstein, Mazkeret Batya (IL); Kent J. Volosin, Mars, PA (US); Rafi Ravid, Savyon (IL)

(21) Appl. No.: 17/656,480

(22) Filed: Mar. 25, 2022

Related U.S. Application Data
(60) Provisional application No. 63/166,580, filed on Mar. 26, 2021.

(51) Int. Cl. A61B 5/021; A61B 5/05; A61B 5/00; A61B 5/282; A61B 5/318

(52) U.S. Cl. CPC A61B 5/02125; A61B 5/05; A61B 5/6823; A61B 5/6824; A61B 5/6833; A61B 5/282; A61B 5/318

(57) ABSTRACT

Medical monitoring systems and techniques for remote monitoring of RF-based and light-based physiological information of a patient are provided. A system as disclosed herein includes an RF transmitter configured to be placed on a predetermined location of the patient and an RF receiver and associated circuitry configured to provide RF sensor signals including information about an RF-based aortic region waveform. The system includes at least one light source configured to be placed on the predetermined location and a light sensor and associated light sensor circuitry configured to provide light sensor signals including information about a light-based arterial waveform. The system includes a processor configured to determine a first fiducial point on the RF-based aortic region waveform, determine a second fiducial point on the light-based arterial waveform, determine a time difference parameter between the fiducial points, and determine at least a pulse wave velocity.

### CROSS-REFERENCE TO RELATED APPLICATION

This non-provisional application claims priority to U.S. Provisional Patent Application Ser. No. 63/166,580, filed on Mar. 26, 2021, titled “SYSTEM FOR USING RADIOFREQUENCY AND LIGHT TO DETERMINE PULSE WAVE VELOCITY,” the entirety of which is hereby incorporated by reference.

### BACKGROUND

The present disclosure relates to directing radiofrequency (RF) and light waves into a patient and using the waves to determine, measure, and/or monitor cardiovascular health of the patient.

A caregiver monitoring a patient's cardiovascular health may want to take cardiovascular measurements, such as blood pressure measurements, of a patient. Taking cardiovascular measurements often requires a caregiver to manually take a patient's cardiovascular measurements using equipment in a caregiver's office. For example, a caregiver may take a patient's blood pressure using a sphygmomanometer. Otherwise, a patient may be required to purchase equipment and take their own cardiovascular measurements.

However, because cardiovascular measurements are manually taken, either by a caregiver or the patient, this limits the amount of cardiovascular measurements that may be taken from the patient. In turn, the limited amount of cardiovascular measurements may provide a caregiver with an incomplete view of the patient's cardiovascular health. This incomplete view may be compounded by the fact that conditions in the caregiver's office, or at the patient's home, when cardiovascular measurements are being taken may not be representative of the patient's day-to-day conditions that affect cardiovascular readings.

### SUMMARY

In one or more examples, a medical monitoring system for remote monitoring of RF-based and light-based physiological information of a patient is provided. The system includes an RF transmitter configured to generate RF waves. The RF transmitter is configured to be placed on a first location of the patient such that the generated RF waves are directed towards an aortic region of the patient including at least one of an aorta or one or more branching arteries proximate to the aorta. The system includes an RF receiver and associated RF circuitry configured to receive RF waves reflected from the aortic region of the patient. The RF circuitry is configured to provide RF sensor signals, based on the received RF waves, including information about an RF-based aortic region waveform of the patient. The system includes at least one light source configured to generate light of one or more predetermined frequencies. The at least one light source is configured to be placed on the first location of the patient such that the generated light is directed towards one or more arteries below skin on a thorax of the patient. The system includes a light sensor and associated light sensor circuitry configured to receive light reflected from the one or more arteries below the skin. The light sensor circuitry is configured to provide light sensor signals, based on the received light, including information about a light-based arterial waveform of the patient. The system includes a memory implemented in a non-transitory media and a processor in communication with the memory. The processor is configured to determine a first fiducial point on the RF-based aortic region waveform, determine a second fiducial point on the light-based arterial waveform, determine a time difference parameter between the first fiducial point and the second fiducial point, and determine, using the time difference parameter and a distance along an arterial tree between the aortic region and the one or more arteries below the skin, a pulse wave velocity of the patient.

Implementations of the medical monitoring system for remote monitoring of RF-based and light-based physiological information of a patient can include one or more of the following features. The first location includes a location on skin above a sternum of the patient. The system includes a second RF transmitter configured to generate a second set of RF waves. The second RF transmitter is configured to be placed on a second location of the patient such that the second set of RF waves are directed towards an artery of the patient at the second location. The system includes a second RF receiver and associated second RF circuitry configured to receive a second set of RF waves reflected from the artery at the second location of the patient. The second RF circuitry is configured to provide a second set of RF signals, based on the received second set of RF waves, including information about an RF-based waveform of the artery at the second location. The processor is further configured to determine a third fiducial point on the RF-based aortic region waveform, determine a fourth fiducial point on the RF-based waveform of the artery at the second location, and determine a second time difference parameter between the third fiducial point and the fourth fiducial point. The processor is further configured to determine, using the second time difference parameter and a distance along the arterial tree between the aortic region and the artery at the second location, a second pulse wave velocity of the patient. The processor is further configured to determine, using the second time difference parameter, a second blood pressure of the patient. The second location includes a location above a radial artery of the patient, and the RF-based waveform of the artery at the second location includes an RF-based radial waveform of the patient. The second location includes a location above a subclavian artery of the patient, and the RF-based waveform of the artery at the second location includes an RF-based subclavian waveform of the patient. The second location includes a location above a brachial artery of the patient, and wherein the RF-based waveform of the artery at the second location includes an RF-based brachial waveform of the patient.

The first fiducial point com includes a local minimum of the RF-based aortic region waveform. The first fiducial point includes a local maximum of the RF-based aortic region waveform. The RF-based aortic region waveform includes at least a primary aortic region peak, and the first fiducial point includes an onset of the primary aortic region peak, an apex of the primary aortic region peak, or an end of the primary aortic region peak. The RF-based aortic region waveform includes at least a primary aortic region peak and a secondary aortic region peak, and the first fiducial point includes an onset of the secondary aortic region peak, an apex of the secondary aortic region peak, or an end of the secondary aortic region peak.

The second fiducial point includes a local minimum of the light-based arterial waveform. The second fiducial point includes a local maximum of the light-based arterial waveform. The light-based arterial waveform includes at least a primary arterial peak, and the second fiducial point includes an onset of the primary arterial peak, an apex of the primary arterial peak, or an end of the primary arterial peak. The light-based arterial waveform includes at least a primary arterial peak and a secondary arterial peak, and the second fiducial point includes an onset of the secondary arterial peak, an apex of the secondary arterial peak, or an end of the secondary arterial peak.

The processor is configured to determine the pulse wave velocity of the patient by dividing the distance along the arterial tree between the aortic region and the one or more arteries below the skin by the time difference parameter. The processor is further configured to receive the distance along the arterial tree between the aortic region and the one or more arteries below the skin from a caregiver. The processor is further configured to determine the distance along the arterial tree between the aortic region and the one or more arteries below the skin based on a body mass index (BMI) of the patient. The RF sensor signals are first RF sensor signals. The RF receiver and associated RF circuitry are further configured to receive RF waves reflected from a posterior of the patient's thorax. The RF circuitry is configured to provide second RF sensor signals based on the RF waves reflected from the posterior of the patient's thorax. The processor is further configured to determine, based on the second RF sensor signals, an anteroposterior diameter of the patient. The processor is further configured to determine the distance along the arterial tree between the aortic region and the one or more arteries below the skin from the anteroposterior diameter.

The processor is further configured to determine, using at least one of the pulse wave velocity or the time difference parameter, a blood pressure of the patient. The processor is configured to determine the blood pressure of the patient based on a predetermined function of the time difference parameter. The predetermined function includes one or a combination of a linear function, an nth-order polynomial function, a logarithmic function, or an exponential function. The time difference parameter includes a pulse transit time (PTT). The processor is configured to determine the blood pressure of the patient based on P=A*ln(PTT)+B. P includes the blood pressure and A and B include pre-calibrated constants. The processor is further configured to receive a plurality of blood pressure measurements for the patient and calibrate A and B to the patient based on the plurality of blood pressure measurements. The processor is configured to determine a systolic blood pressure of the patient based on the formula Pₛ=C*ln(PTT)+D and determine a diastolic blood pressure of the patient based on the formula P_{d}=F*ln(PTT)+G, wherein Pₛ includes the systolic blood pressure, P_{d} includes the diastolic blood pressure, and C, D, F, and G include pre-calibrated constants. The processor is further configured to receive a plurality of blood pressure measurements for the patient and calibrate C, D, F, and G to the patient based on the plurality of blood pressure measurements. The processor is configured to determine the blood pressure of the patient based on a predetermined function of a logarithm of a square of the pulse wave velocity. The processor is configured to determine the blood pressure of the patient further using one or more pre-calibrated constants. The processor is further configured to receive one or more control blood pressure measurements of the patient and calibrate the one or more constants based on the one or more control blood pressure measurements.

The time difference parameter between the first fiducial point and the second fiducial point is one of a plurality of time difference parameters between fiducial points of the RF-based aortic region waveform and light-based arterial waveform over a summary time period. The processor is further configured to determine the plurality of time difference parameters by determining a plurality of first fiducial points on the RF-based aortic region waveform, determining a plurality of second fiducial points on the light-based arterial waveform, and determining a time difference parameter between each first fiducial point and corresponding second fiducial point. Each of the plurality of time difference parameters corresponds to a cardiac cycle of the patient occurring in the summary time period. The summary time period includes at least one of 3-5 cardiac cycles, 5-10 cardiac cycles, 10-15 cardiac cycles, or 15-20 cardiac cycles. The processor is further configured to determine, using the plurality of time difference parameters, a summary time difference parameter for the summary time period. The summary time difference parameter includes a mean, a median, a mode, a minimum, a maximum, or another statistical measure of the plurality of time difference parameters. The processor is further configured to determine, using the plurality of time difference parameters and the distance along the arterial tree between the aortic region and the one or more arteries below the skin, a summary pulse wave velocity of the patient for the summary time period. The summary pulse wave velocity includes a mean pulse wave velocity, a median pulse wave velocity, a mode pulse wave velocity, a minimum pulse wave velocity, a maximum pulse wave velocity, or another statistical measure of pulse wave velocity for the summary time period.

The system includes a patch configured to be adhesively attached to the first location of the patient. The RF transmitter and the RF receiver and associated RF circuitry are configured to be mounted onto the patch. The at least one light source and the light sensor are embedded into the patch. At least a portion of the patch is transparent, and wherein the at least one light source and the light sensor are configured to be mounted onto the patch over the transparent portion. The system includes a band configured to wrap around the thorax of the patient. The RF transmitter, the RF receiver and associated RF circuitry, the at least one light source, and the light sensor and associated light sensor circuitry are configured to be mounted onto the band. The at least one light source includes at least one diode. The system includes two or more ECG electrodes. The processor is further configured to receive ECG signals from the two or more ECG electrodes.

The system includes a monitoring device. The monitoring device includes the memory, the processor, and at least some of the RF transmitter, the RF receiver and associated RF circuitry, the at least one light source, or the light sensor and associated light sensor circuitry. The system includes a remote server. The remote server includes the memory and the processor.

In one or more examples, a medical monitoring system for remote monitoring of RF-based and light-based physiological information of a patient is provided. The system includes a monitoring device, which includes an RF transmitter configured to generate RF waves. The RF transmitter is configured to be place on a first location of the patient such that the generated RF waves are directed towards an aortic region of the patient including at least one of an aorta or one or more branching arteries proximate to the aorta. The monitoring device includes an RF receiver and associated RF circuitry configured to receive RF waves reflected from the aorta of the patient. The RF circuitry is configured to provide RF sensor signals, based on the received RF waves, including information about an RF-based aortic region waveform of the patient. The monitoring device includes at least one light source configured to generate light of one or more predetermined frequencies. The at least one light source is configured to be placed on the first location of the patient such that the generated light is directed towards one or more arteries below skin on a thorax of the patient. The monitoring device includes a light sensor and associated light sensor circuitry configured to receive light reflected from the one or more arteries below the skin. The light sensor circuitry is configured to provide light sensor signals, based on the received light, including information about a light-based arterial waveform of the patient. The monitoring device is configured to transmit the RF sensor signals and the light sensor signals to a remote server. The system includes he remote server in communication with the monitoring device. The remote server includes a database implemented in non-transitory media and a processor in communication with the database. The processor is configured to determine a first fiducial point on the RF-based aortic region waveform, determine a second fiducial point on the light-based arterial waveform, determine a time difference parameter between the first fiducial point and the second fiducial point, and determine, using the time difference parameter and a distance along an arterial tree between the aortic region and the one or more arteries below the skin, a pulse wave velocity of the patient.

Implementations of the medical monitoring system for remote monitoring of RF-based and light-based physiological information of a patient can include one or more of the following features. The first location includes a location on skin above a sternum of the patient. The system includes a second RF transmitter configured to generate a second set of RF waves. The second RF transmitter is configured to be placed on a second location of the patient such that the second set of RF waves are directed towards an artery of the patient at the second location. The system includes a second RF receiver and associated second RF circuitry configured to receive a second set of RF waves reflected from the artery at the second location of the patient. The second RF circuitry is configured to provide a second set of RF signals, based on the received second set of RF waves, including information about an RF-based waveform of the artery at the second location. The processor is further configured to determine a third fiducial point on the RF-based aortic region waveform, determine a fourth fiducial point on the RF-based waveform of the artery at the second location, and determine a second time difference parameter between the third fiducial point and the fourth fiducial point. The processor is further configured to determine, using the second time difference parameter and a distance along the arterial tree between the aortic region and the artery at the second location, a second pulse wave velocity of the patient. The processor is further configured to determine, using the second time difference parameter, a second blood pressure of the patient. The second location includes a location above a radial artery of the patient, and the RF-based waveform of the artery at the second location includes an RF-based radial waveform of the patient. The second location includes a location above a subclavian artery of the patient, and the RF-based waveform of the artery at the second location includes an RF-based subclavian waveform of the patient. The second location includes a location above a brachial artery of the patient, and wherein the RF-based waveform of the artery at the second location includes an RF-based brachial waveform of the patient.

The first fiducial point com includes a local minimum of the RF-based aortic region waveform. The first fiducial point includes a local maximum of the RF-based aortic region waveform. The RF-based aortic region waveform includes at least a primary aortic region peak, and the first fiducial point includes an onset of the primary aortic region peak, an apex of the primary aortic region peak, or an end of the primary aortic region peak. The RF-based aortic region waveform includes at least a primary aortic region peak and a secondary aortic region peak, and the first fiducial point includes an onset of the secondary aortic region peak, an apex of the secondary aortic region peak, or an end of the secondary aortic region peak.

The second fiducial point includes a local minimum of the light-based arterial waveform. The second fiducial point includes a local maximum of the light-based arterial waveform. The light-based arterial waveform includes at least a primary arterial peak, and the second fiducial point includes an onset of the primary arterial peak, an apex of the primary arterial peak, or an end of the primary arterial peak. The light-based arterial waveform includes at least a primary arterial peak and a secondary arterial peak, and the second fiducial point includes an onset of the secondary arterial peak, an apex of the secondary arterial peak, or an end of the secondary arterial peak.

The processor is configured to determine the pulse wave velocity of the patient by dividing the distance along the arterial tree between the aortic region and the one or more arteries below the skin by the time difference parameter. The processor is further configured to receive the distance along the arterial tree between the aortic region and the one or more arteries below the skin from a caregiver. The processor is further configured to determine the distance along the arterial tree between the aortic region and the one or more arteries below the skin based on a BMI of the patient. The RF sensor signals are first RF sensor signals. The RF receiver and associated RF circuitry are further configured to receive RF waves reflected from a posterior of the patient's thorax. The RF circuitry is configured to provide second RF sensor signals based on the RF waves reflected from the posterior of the patient's thorax. The processor is further configured to determine, based on the second RF sensor signals, an anteroposterior diameter of the patient. The processor is further configured to determine the distance along the arterial tree between the aortic region and the one or more arteries below the skin from the anteroposterior diameter.

The processor is further configured to determine, using at least one of the pulse wave velocity or the time difference parameter, a blood pressure of the patient. The processor is configured to determine the blood pressure of the patient based on a predetermined function of the time difference parameter. The predetermined function includes one or a combination of a linear function, an nth-order polynomial function, a logarithmic function, or an exponential function. The time difference parameter includes a pulse transit time (PTT). The processor is configured to determine the blood pressure of the patient based on P=A*ln(PTT)+B. P includes the blood pressure and A and B include pre-calibrated constants. The processor is further configured to receive a plurality of blood pressure measurements for the patient and calibrate A and B to the patient based on the plurality of blood pressure measurements. The processor is configured to determine a systolic blood pressure of the patient based on the formula Pₛ=C*ln(PTT)+D and determine a diastolic blood pressure of the patient based on the formula P_{d}=F*ln(PTT)+G, wherein Pₛ includes the systolic blood pressure, P_{d} includes the diastolic blood pressure, and C, D, F, and G include pre-calibrated constants. The processor is further configured to receive a plurality of blood pressure measurements for the patient and calibrate C, D, F, and G to the patient based on the plurality of blood pressure measurements. The processor is configured to determine the blood pressure of the patient based on a predetermined function of a logarithm of a square of the pulse wave velocity. The processor is configured to determine the blood pressure of the patient further using one or more pre-calibrated constants. The processor is further configured to receive one or more control blood pressure measurements of the patient and calibrate the one or more constants based on the one or more control blood pressure measurements.

The time difference parameter between the first fiducial point and the second fiducial point is one of a plurality of time difference parameters between fiducial points of the RF-based aortic region waveform and light-based arterial waveform over a summary time period. The processor is further configured to determine the plurality of time difference parameters by determining a plurality of first fiducial points on the RF-based aortic region waveform, determining a plurality of second fiducial points on the light-based arterial waveform, and determining a time difference parameter between each first fiducial point and corresponding second fiducial point. Each of the plurality of time difference parameters corresponds to a cardiac cycle of the patient occurring in the summary time period. The summary time period includes at least one of 3-5 cardiac cycles, 5-10 cardiac cycles, 10-15 cardiac cycles, or 15-20 cardiac cycles. The processor is further configured to determine, using the plurality of time difference parameters, a summary time difference parameter for the summary time period. The summary time difference parameter includes a mean, a median, a mode, a minimum, a maximum, or another statistical measure of the plurality of time difference parameters. The processor is further configured to determine, using the plurality of time difference parameters and the distance along the arterial tree between the aortic region and the one or more arteries below the skin, a summary pulse wave velocity of the patient for the summary time period. The summary pulse wave velocity includes a mean pulse wave velocity, a median pulse wave velocity, a mode pulse wave velocity, a minimum pulse wave velocity, a maximum pulse wave velocity, or another statistical measure of pulse wave velocity for the summary time period.

The system includes a patch configured to be adhesively attached to the first location of the patient. The RF transmitter and the RF receiver and associated RF circuitry are configured to be mounted onto the patch. The at least one light source and the light sensor are embedded into the patch. At least a portion of the patch is transparent, and wherein the at least one light source and the light sensor are configured to be mounted onto the patch over the transparent portion. The system includes a band configured to wrap around the thorax of the patient. The RF transmitter, the RF receiver and associated RF circuitry, the at least one light source, and the light sensor and associated light sensor circuitry are configured to be mounted onto the band. The at least one light source includes at least one diode. The system includes two or more ECG electrodes. The processor is further configured to receive ECG signals from the two or more ECG electrodes.

In one or more examples, a method for remote monitoring of RF-based and light-based physiological information of a patient is provided. The method includes generating, by an RF transmitter, RF waves directed towards an aortic region of the patient including at least one of an aorta or one or more branching arteries proximate to the aorta; receiving, by an RF receiver and associated RF circuitry, RF waves reflected from the aortic region of the patient; and providing, by RF circuitry, RF sensor signals based on the RF waves. The RF sensor signals include information about an RF-based aortic region waveform of the patient. The method includes generating, by at least one light source, light of one or more predetermined frequencies directed towards one or more arteries below skin on a thorax of the patient; receiving, by a light sensor and associated light sensor circuitry, light reflected from the one or more arteries below the skin; and providing, by the light sensor circuitry, light sensor signals based on the received light. The light sensor signals include information about a light-based arterial waveform of the patient. The method includes determining a first fiducial point on the RF-based aortic region waveform, determining a second fiducial point on the light-based arterial waveform, determining a time difference parameter between the first fiducial point and the second fiducial point, and determining, using the time difference parameter and a distance along an arterial tree between the aortic region and the one or more arteries below the skin, a pulse wave velocity of the patient.

Implementations of the method for remote monitoring of RF-based and light-based physiological information of a patient can include one or more of the following features. The RF transmitter, the RF receiver and associated circuitry, the at least one light source, and the light sensor and associated light sensor circuitry are configured to be placed on a first location of the patient. The first location includes a location on skin above a sternum of the patient. The method includes generating, by a second RF transmitter, a second set of RF waves directed towards an artery of the patient at a second location of the patient; receiving, by a second RF receiver and associated second RF circuitry, a second set of RF waves reflected from the artery at the second location of the patient; and providing, by the second RF circuitry, a second set of RF signals based on the received second set of RF waves. The second set of RF signals include information about an RF-based waveform of the artery at the second location. The method includes determining a third fiducial point on the RF-based aortic region waveform, determining a fourth fiducial point on the RF-based waveform of the artery at the second location, and determining a second time difference parameter between the third fiducial point and the fourth fiducial point. The method includes determining, using the second time difference parameter and a distance along the arterial tree between the aortic region and the artery at the second location, a second pulse wave velocity of the patient. The method includes determining, using the second time difference parameter, a second blood pressure of the patient. The second location includes a location above a radial artery of the patient, and the RF-based waveform of the artery at the second location includes an RF-based radial waveform of the patient. The second location includes a location above a subclavian artery of the patient, and the RF-based waveform of the artery at the second location includes an RF-based subclavian waveform of the patient. The second location includes a location above a brachial artery of the patient, and the RF-based waveform of the artery at the second location includes an RF-based brachial waveform of the patient.

The first fiducial point includes a local minimum of the RF-based aortic region waveform. The first fiducial point includes a local maximum of the RF-based aortic region waveform. The RF-based aortic region waveform includes at least a primary aortic region peak, and the first fiducial point includes an onset of the primary aortic region peak, an apex of the primary aortic region peak, or an end of the primary aortic region peak. The RF-based aortic region waveform includes at least a primary aortic region peak and a secondary aortic region peak, and the first fiducial point includes an onset of the secondary aortic region peak, an apex of the secondary aortic region peak, or an end of the secondary aortic region peak. The second fiducial point includes a local minimum of the light-based arterial waveform. The second fiducial point includes a local maximum of the light-based arterial waveform. The light-based arterial waveform includes at least a primary arterial peak, and the second fiducial point includes an onset of the primary arterial peak, an apex of the primary arterial peak, or an end of the primary arterial peak. The light-based arterial waveform includes at least a primary arterial peak and a secondary arterial peak, and the second fiducial point includes an onset of the secondary arterial peak, an apex of the secondary arterial peak, or an end of the secondary arterial peak.

Determining the pulse wave velocity of the patient includes dividing the distance along the arterial tree between the aortic region and the one or more arteries below the skin by the time difference parameter. The method includes receiving the distance along the arterial tree between the aortic region and the one or more arteries below the skin from a caregiver. The method includes determining the distance along the arterial tree between the aortic region and the one or more arteries below the skin based on an BMI of the patient. The RF sensor signals are first RF sensor signals. The method further receiving, by the RF receiving and associated RF circuitry, RF waves reflected from a posterior of the patient's thorax; providing, by the RF circuitry, second RF sensor signals based on the RF waves reflected from the posterior of the patient's thorax; and determining, based on the second RF sensor signals, an anteroposterior diameter of the patient. The method includes determining the distance along the arterial tree between the aortic region and the one or more arteries below the skin from the anteroposterior diameter.

The method includes determining, using at least one of the pulse wave velocity or the time difference parameter, a blood pressure of the patient. Determining the blood pressure of the patient includes determining the blood pressure of the patient based on a predetermined function of the time difference parameter. The predetermined function includes one or a combination of a linear function, an nth-order polynomial function, a logarithmic function, or an exponential function. The time difference parameter includes a pulse transit time (PTT), wherein determining the blood pressure of the patient includes determining the blood pressure of the patient based on based on P=A*ln(PTT)+B, and wherein P includes the blood pressure and A and B include pre-calibrated constants. The method includes receiving a plurality of blood pressure measurements for the patient and calibrating A and B to the patient based on the plurality of blood pressure measurements. Determining the blood pressure of the patient includes determining a systolic blood pressure of the patient based on the formula Pₛ=C*ln(PTT)+D and determining a diastolic blood pressure of the patient based on the formula P_{d}=F*ln(PTT)+G. Pₛ includes the systolic blood pressure, P_{d} includes the diastolic blood pressure, and C, D, F, and G include pre-calibrated constants. The method of claim 125 includes receiving a plurality of blood pressure measurements for the patient and calibrating C, D, F, and G to the patient based on the plurality of blood pressure measurements. Determining the blood pressure of the patient includes determining the blood pressure of the patient based on a predetermined function of a logarithm of a square of the pulse wave velocity. Determining the blood pressure of the patient includes determining the blood pressure of the patient using one or more pre-calibrated constants. The method of claim 128, includes receiving one or more control blood pressure measurements of the patient and calibrating the one or more constants based on the one or more control blood pressure measurements.

The time difference parameter between the first fiducial point and the second fiducial point is one of a plurality of time difference parameters between fiducial points of the RF-based aortic region waveform and light-based arterial waveform over a summary time period. The method includes determining the plurality of time difference parameters by determining a plurality of first fiducial points on the RF-based aortic region waveform, determining a plurality of second fiducial points on the light-based arterial waveform, and determining a time difference parameter between each first fiducial point and corresponding second fiducial point. Each of the plurality of time difference parameters corresponds to a cardiac cycle of the patient occurring in the summary time period. The summary time period includes at least one of 3-5 cardiac cycles, 5-10 cardiac cycles, 10-15 cardiac cycles, or 15-20 cardiac cycles. The method includes determining, using the plurality of time difference parameters, a summary time difference parameter for the summary time period. The summary time difference parameter includes a mean, a median, a mode, a minimum, a maximum, or another statistical measure of the plurality of time difference parameters. The method includes determining, using the plurality of time difference parameters and the distance along the arterial tree between the aortic region and the one or more arteries below the skin, a summary pulse wave velocity for the patient for the summary time period. The summary pulse wave velocity includes a mean pulse wave velocity, a median pulse wave velocity, a mode pulse wave velocity, a minimum pulse wave velocity, a maximum pulse wave velocity, or another statistical measure of pulse wave velocity for the summary time period.

The RF transmitter and the RF receiver and associated circuitry are configured to be mounted onto a patch configured to be adhesively attached to a first location of the patient. The at least one light source and the light sensor are embedded into the patch. At least a portion of the patch is transparent. The at least one light source and the light sensor are configured to be mounted onto the patch over the transparent portion. The RF transmitter, the RF receiver and associated RF circuitry, the at least one light source, and the light sensor and associated light sensor circuitry are configured to be mounted onto a band configured to wrap around the thorax of the patient. The at least one light source includes at least one diode. The method includes receiving ECG signals from two or more ECG electrodes.

A monitoring device includes a memory implemented in a non-transitory media, a processor in communication with the memory, and at least some of the RF transmitter, the RF receiver and associated RF circuitry, the at least one light source, or the light sensor and associated light sensor circuitry. Determining the first fiducial point on the RF-based aortic region waveform includes determining, by the processor of the monitoring device, the first fiducial point on the RF-based aortic region waveform. Determining the second fiducial point on the light-based arterial waveform includes determining, by the processor of the monitoring device, the second fiducial point on the light-based arterial waveform. Determining the time difference parameter between the first fiducial point and the second fiducial point includes determining, by the processor of the monitoring device, the time difference parameter between the first fiducial point and the second fiducial point. Determining, using the time difference parameter and the distance along the arterial tree between the aortic region and the one or more arteries below the skin, the pulse wave velocity of the patient includes determining, by the processor of the monitoring device, using the time difference parameter and the distance along the arterial tree between the aortic region and the one or more arteries below the skin, the pulse wave velocity of the patient.

Determining the first fiducial point on the RF-based aortic region waveform includes determining, by a processor of a remote server, the first fiducial point on the RF-based aortic region waveform. Determining the second fiducial point on the light-based arterial waveform includes determining, by the processor of the remote server, the second fiducial point on the light-based arterial waveform. Determining the time difference parameter between the first fiducial point and the second fiducial point includes determining, by the processor of the remote server, the time difference parameter between the first fiducial point and the second fiducial point. Determining, using the time difference parameter and the distance along the arterial tree between the aortic region and the one or more arteries below the skin, the pulse wave velocity of the patient includes determining, by the processor of the remote server, using the time difference parameter and the distance along the arterial tree between the aortic region and the one or more arteries below the skin, the pulse wave velocity of the patient.

In one or more examples, a medical monitoring system for remote monitoring of radiofrequency (RF)-based and light-based physiological information of a patient is provided. The system includes an RF transmitter configured to generate RF waves. The RF transmitter is configured to be placed on a predetermined location of the patient such that the generated RF waves are directed towards an aortic region of the patient including at least one of an aorta or one or more branching arteries proximate to the aorta. The system includes an RF receiver and associated RF circuitry configured to receive RF waves reflected from the aortic region of the patient. The RF circuitry is configured to provide RF sensor signals, based on the received RF waves, including information about an RF-based aortic region waveform of the patient. The system includes at least one light source configured to generate light of one or more predetermined frequencies. The at least one light source is configured to be placed on the predetermined location of the patient such that the generated light is directed towards one or more arteries below skin on a thorax of the patient. The system includes a light sensor and associated light sensor circuitry configured to receive light reflected from the one or more arteries below the skin. The light sensor circuitry is configured to provide light sensor signals, based on the received light, including information about a light-based arterial waveform of the patient. The system includes a memory implemented in a non-transitory media and a processor in communication with the memory. The processor is configured to determine a first fiducial point on the RF-based aortic region waveform, determine a second fiducial point on the light-based arterial waveform, and determine a time difference parameter between the first fiducial point and the second fiducial point.

Implementations of the medical monitoring system for remote monitoring of radiofrequency (RF)-based and light-based physiological information of a patient can include one or more of the following features. The processor is configured to determine, using the time difference parameter and a distance along an arterial tree between the aortic region and the one or more arteries below the skin, a pulse wave velocity of the patient. The processor is configured to determine the pulse wave velocity of the patient by dividing the distance along the arterial tree between the aortic region and the one or more arteries below the skin by the time difference parameter. The processor is further configured to receive the distance along the arterial tree between the aortic region and the one or more arteries below the skin from a caregiver. The processor is configured to determine the blood pressure of the patient based on a predetermined function of the time difference parameter. The predetermined function includes one or a combination of a linear function, an nth-order polynomial function, a logarithmic function, or an exponential function.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example medical monitoring system.

FIG. 2 depicts an example adhesive patch.

FIG. 3 depicts an example monitoring device.

FIG. 4 depicts an example of a monitoring device being attached to an adhesive patch.

FIG. 5 depicts an example exploded view of a monitoring device.

FIG. 6 depicts an example electronic architecture for a monitoring device.

FIG. 7 depicts an example electronic architecture for RF functionality of a monitoring device.

FIG. 8 depicts an example adhesive patch and monitoring device positioned on a patient.

FIG. 9 depicts another example monitoring device.

FIG. 10 depicts another example adhesive patch.

FIG. 11 depicts an example band and monitoring device positioned on a patient.

FIG. 12 depicts an example wearable combination piece and monitoring device positioned on a patient.

FIG. 13 depicts another example wearable combination piece and monitoring device positioned on a patient.

FIG. 14 depicts another example adhesive patch and monitoring device positioned on a patient.

FIG. 15 depicts another example adhesive patch and monitoring device positioned on a patient.

FIG. 16 depicts an example adhesive patch, band, and monitoring device positioned on a patient.

FIG. 17 depicts an example monitoring device with sensor patches positioned on a patient.

FIG. 18 depicts an example garment-based medical device and monitoring device positioned on a patient.

FIG. 19 depicts an example electronic architecture for a medical device controller of a garment-based medical device.

FIG. 20 depicts an example process flow of providing RF and light sensor signals.

FIG. 21A depicts an example of a cardiovascular monitoring unit being used on a patient.

FIG. 21B depicts an example aortic region of a patient.

FIG. 22 depicts an example aortic region waveform over time.

FIG. 23 depicts an example arterial waveform over time.

FIG. 24 depicts an example process flow of determining a cardiovascular measurement.

FIG. 25 depicts an example RF sensor waveform, light sensor waveform, and ECG waveform over time.

FIG. 26 depicts example aortic region and/or arterial waveforms.

FIG. 27 depicts an example process flow of determining a patient's blood pressure.

FIG. 28 depicts an example adhesive patch, monitoring device, and armband device positioned on a patient.

FIG. 29 depicts an example radial waveform over time.

FIG. 30 depicts an example process flow of determining a cardiovascular measurement.

FIG. 31 depicts an example adhesive patch, monitoring device, and armband device positioned on a patient.

FIG. 32 depicts an example adhesive patch, monitoring device, secondary adhesive patch, and secondary monitoring device positioned on a patient.

FIG. 33 depicts another example adhesive patch, monitoring device, secondary adhesive patch, and secondary monitoring device positioned on a patient.

FIG. 34 depicts example RF-based aortic region, subclavial, and radial waveforms and an example light-based arterial waveform.

FIG. 35 depicts an example ECG waveform and example RF-based aortic region, subclavial, and radial waveforms.

### DETAILED DESCRIPTION

In a cardiology practice, a caregiver may want to determine, measure, and/or monitor the condition of a patient's circulatory system. As such, a caregiver may take measurements relating to the patient's blood pressure or other arterial characteristics, such as pulse wave velocity, from the patient that represent the status of the patient's circulatory system. However, one of the most commonly taken circulatory measurements is blood pressure, and blood pressure is typically measured through a sphygmomanometer. The sphygmomanometer applies pressure to a patient's blood vessel, causing the blood vessel to constrict. The sphygmomanometer then slowly releases the pressure on the blood vessel, monitoring the pressure still applied to the blood vessel as it relaxes. Accordingly, measuring a patient's blood pressure using a sphygmomanometer involves a process that is physically performed on the patient. Thus, the patient typically travels to the location of a caregiver, who manually takes the patient's blood pressure. Because the patient is traveling to the sphygmomanometer, there are limited times when the patient's blood pressure can be taken. Otherwise, the patient can purchase a sphygmomanometer and manually take their own blood pressure. This allows for more frequent blood pressure measurements but depends on the patient being able to reliably take these measurements. Additionally, for a caregiver to see the measurements, the patient must typically provide them to the caregiver, such as by recording them in a patient journal that the patient provides to the caregiver as their next appointment. Because the patient is providing the measurements to the provider, there is inherently a delay between when the measurements are taken and when the provider can review the measurements.

This disclosure relates to an improved medical monitoring system that remotely determines, measures, and/or monitors a condition of the patient's circulatory system. A patient may be prescribed a monitoring device configured to be worn continuously for an extended period of time. The monitoring device incorporates both an RF transmitter and receiver and a light source and sensor. As such, the monitoring device automatically monitors blood vessels of the patient's circulatory system using RF and light waves. The monitoring device further generates RF signals and light signals that include information about waveforms of the monitored blood vessels, where the waveforms correspond to the pulse pressure of the monitored blood vessels. The monitoring device may transmit (e.g. via communications circuitry that are internal to the monitoring device, or communications circuitry within a separate portable gateway) the RF signals and light signals to a remote server. In some implementations, the monitoring device may determine one or more circulatory measurements from the RF and light signals and transmit the circulatory measurements to the remote server in addition to, or in the alternative from, transmitting the RF and light signals. In some implementations, the remote server may determine one or more circulatory measurements from the RF and light signals received from the monitoring device. The remote server then provides circulatory measurements and/or a summary of the circulatory measurements to a caregiver of the patient.

For instance, in some implementations, a medical monitoring system may provide for remote monitoring of RF-based and light-based physiological information of a patient, as noted above. The medical monitoring system may include a monitoring device that includes an RF transmitter, as well as an RF receiver and associated circuitry, such that the monitoring device may direct RF waves towards an aortic region of a patient and receive reflected RF waves from the aortic region. For example, the aortic region includes the patient's aorta and/or one or more arteries branching off from and proximate to the aorta. At the same physiological location (e.g., on the sternum), the monitoring device may further include at least one light source and a light sensor and associated circuitry such that the monitoring device may direct light towards arteries below skin on the thorax of the patient and receive reflected light waves from the arteries. For instance, the monitoring device may be positioned over or near the sternum of a patient (e.g., over or near the manubrium, over or near the sternal angle, over or near the body of the sternum between the second and third costal notches, over or near the body of the sternum between the third and fourth costal notches, over or near the body of the sternum between the fourth and fifth costal notches, over or near the body of the sternum between the fifth and sixth costal notches, and so on). Positioning the monitoring device over or near the sternum may allow the monitoring device to simultaneously direct RF waves to the aortic region and light waves to the arteries above the sternum.

Based on the RF waves reflected from the aortic region, the monitoring device may provide RF sensor signals that include information about an aortic region waveform. The aortic region waveform corresponds with the volume of the aorta and/or arteries branching off from the aorta over time. The aorta and/or branching arteries expand when a ventricular contraction of the heart occurs, ejecting blood into the aorta (and, from the aorta, into the arteries branching off of the aorta), and contract when the ventricular ejection is finished. Similarly, based on the light waves reflected from the surface arteries, the monitoring device may provide light sensor signals that include information about an arterial waveform, which also corresponds with the volume of the surface arteries over time. The surface arteries also expand after a ventricular ejection. However, because the aortic region is adjacent to the heart and the surface arteries are a distance along the patient's arterial tree from the heart, there is a delay between this pulse wave that occurs at the aortic region and the pulse wave that occurs at the surface arteries. As such, the monitoring device or a remote server in communication with the monitoring device may identify a first fiducial point of the pulse wave on the aortic region waveform and a corresponding second fiducial point of the pulse wave on the arterial waveform. The monitoring device or remote server may then determine a time difference between the two fiducial points (e.g., the pulse transit time). Using this time difference, the monitoring device or remote server may further determine one or more measurements that represent the status of the patient's circulatory system, such as the pulse arrival time, pulse wave velocity and/or the blood pressure.

In one example use case, a caregiver may prescribe that a patient with cardiovascular issues wear a monitoring device for a certain amount of time (e.g., 15 days, 30 days, 60 days, 90 days). The monitoring device is configured to be positioned over the patient's sternum and may take periodic measurements indicative of the patient's cardiovascular health over that time period. For instance, the monitoring device may take one or more measurements every day while the patient is sleeping (e.g., as determined based on the time of day and/or based on accelerometer signals from an accelerometer incorporated in the monitoring device). The monitoring device may take the one or more measurements by directing RF waves towards the patient's aortic region and receiving reflected RF waves to produce an aortic region waveform for the patient, and by directing light towards one or more arteries over the patient's sternum and receiving reflected light to produce an arterial waveform for the patient. The monitoring device may transmit RF sensor signals that include information indicative of the aortic region waveform and light sensor signals that include information indicative of the arterial waveform to a remote server. The remote server analyzes the aortic region waveform and the arterial waveform to determine one or more cardiovascular measurements for the patient, such as the time it takes a pulse wave to travel from the aortic region to the one or more arteries over the patient's sternum (e.g., pulse transit time) and/or the patient's blood pressure. In some instances, the remote server may prepare a report summarizing the cardiovascular measurement(s) and transmit the report to the caregiver.

In another example use case, a patient may complain to a caregiver that the patient has been suffering from a shortness of breath. The caregiver may prescribe a monitoring device and an adhesive patch for the patient to wear for a certain amount of time. In some implementations, the adhesive patch removably attaches to the patient and includes ECG electrodes. The monitoring device removably couples, connects, or snaps into the adhesive patch to receive ECG signals from the ECG electrodes. Alternatively, the monitoring device includes the ECG electrodes, and the adhesive patch includes hydrogen layers configured to interface the ECG electrodes with the patient's skin. In such implementations, the monitoring device removably couples, connects, or snaps into the adhesive patch to begin receiving ECG signals. The monitoring device further produces RF sensor signals from RF waves reflected from the patient's aortic region and light sensor signals from light waves reflected from the patient's sternum, as described above. The monitoring device may transmit the ECG signals, RF sensor signals, and light sensor signals to a remote server. The remote server uses the signals to determine whether the patient experiences any arrhythmias (e.g., from the ECG signals) and the patient's blood pressure over time (e.g., from the RF and light sensor signals). At the end of the use period, the remote server prepares a report for the caregiver summarizing whether the patient experienced any arrhythmias and the patient's blood pressure over time.

In another example use case, a patient may be at high risk for a cardiac event (e.g., a life-threatening cardiac arrhythmia). As such, the patient's caregiver may prescribe that the patient continuously wear a garment-based medical device until the patient is scheduled for a surgery to receive an implantable defibrillator. The patient may wear the garment-based medical device (e.g., shaped like a vest), which monitors for potential arrhythmias in the patient via sensing ECG electrodes. If a life-threatening cardiac arrhythmia is detected, the garment-based medical device charges therapeutic electrodes that provide a shock to the patient. In examples, such a garment-based medical device is configured to include a monitoring device that produces RF sensor signals and light sensor signals in the manner described above. The monitoring device uses the RF sensor signals and light sensor signals to continuously or nearly continuously determine the patient's blood pressure and/or arterial characteristics, and activates an alert when the patient's blood pressure and/or arterial characteristics drops too low or is too high (e.g., as measured by an absolute value or a relative departure from a baseline for the patient), or otherwise transgresses certain thresholds. For example, such thresholds can be set by a caregiver and customized to the patient's monitoring and/or treatment plan. The alert may be transmitted directly to the patient (e.g., a vibratory alert, an auditory alert, a visual alert, an alert sent to a personal device of the patient, an alert sent to a portable gateway used by the garment-based medical device and/or monitoring device to communicate with the remote server, or a combination of one or more of such alerts). Alternatively, or additionally, the alert may be transmitted to the patient's caregiver and/or a loved one (e.g., via a smartphone app installed on the loved one's personal handheld device).

The medical monitoring system described herein provides several advantages over prior art systems. For example, the medical monitoring system allows for remote monitoring of a patient's circulatory system. As such, the medical monitoring system may take more measurements relating to a patient's circulatory system than could be accomplished, for instance, by the patient going to a caregiver's office to have their blood pressure monitored or by taking blood pressure measurements themselves. Additionally, because the medical monitoring system provides for remote monitoring of a patient's circulatory system, the remote server may provide real-time or near real-time (e.g., daily and/or weekly) updates on the status of the patient's circulatory system to the patient's caregiver. Furthermore, other devices that can remotely take measurements relating to a patient's circulatory system are typically invasive, requiring implantation into or near a patient's heart or blood vessels. By contrast, the medical monitoring system described herein remotely monitors a patient's circulatory system using RF and light waves that are applied to the patient's body externally through a non-invasive process. In scenarios, the devices and techniques described herein provide more frequent measurements relative to conventional systems and techniques. Accordingly, caregivers and/or a patient's loved ones are able to respond to rapid changes in the patient's underlying cardiovascular health.

In addition, the medical device described herein incorporates an RF transmitter and receiver and a light source and sensor used to monitor the patient's circulatory system into a single, wearable unit. Accordingly, a patient instructed to use the monitoring device does not need to wear, or otherwise apply to their skin, multiple components in multiple locations. Rather, the patient can wear the medical device at a single location, which simplifies the ease of use for a patient. The simplified ease of use also increases the likelihood, for example, that the patient will follow a caregiver's instructions to continuously wear the medical device for an extended period of time. Alternatively, or additionally, the monitoring device may only need to be applied to the patient's skin for a limited period of time (e.g., held to the patient's chest for a few minutes). As such, the fact that the monitoring device is implemented as a single unit may provide easy use for a caregiver or the patient, given that the caregiver or patient need only hold the monitoring device in place on the patient's skin for the limited period of time.

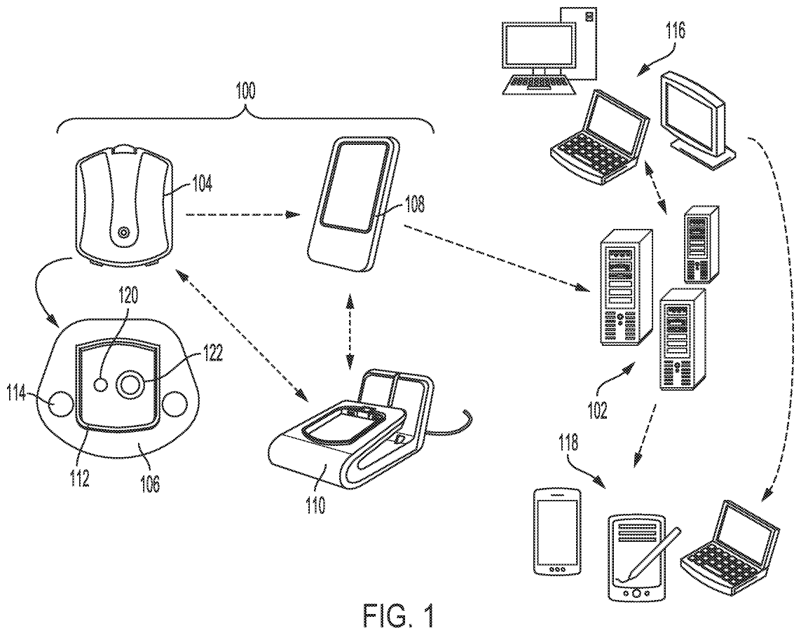

FIG. 1 shows a medical monitoring system that includes a cardiovascular monitoring unit 100 in communication with a remote server 102. The monitoring unit 100 is configured to provide RF sensor signals and light sensor signals that include information about cardiovascular waveforms (e.g., waveforms representing the volumes of the arteries in the patient's aortic region and the patient's surface arteries over time) to the remote server 102. In some embodiments, the cardiovascular monitoring unit 100 includes a cardiovascular monitoring device 104, an adhesive patch 106, a portable gateway 108, and a charger 110. The monitoring device 104 is configured to transmit RF and light waves into a patient and receive reflected RF and light waves from the patient. The monitoring device 104 is further configured to generate the RF sensor signals and light sensor signals using the received RF and light waves and transmit the RF and light sensor signals to the portable gateway 108. In some embodiments, the cardiovascular monitoring unit 100 may include and/or be at least partially implemented by the μCor™ Heart Failure and Arrhythmia Management System (HFAMS) available from ZOLL® Medical Corporation of Chelmsford, Mass.

The adhesive patch 106 is configured to be adhesively coupled to the skin of a patient, where the monitoring device 104 is configured to be removably attached to the adhesive patch 106. For example, the adhesive patch may include a frame 112 in the same general shape of the monitoring device 104, and the monitoring device 104 is configured to removably couple, connect, or snap into the frame 112.

Together, the monitoring device 104 and the adhesive patch 106 include an RF transmitter and an RF receiver, as well as at least one light source and a light sensor. In some embodiments, the adhesive patch 106 may include at least some of the RF transmitter, RF receiver, at least one light source, and light sensor. As an illustration, in the example of FIG. 1, a light source 120 and a light sensor 122 may be embedded into the adhesive patch such that the light source 120 and the light sensor 122 face the skin of the patient. The monitoring device 104 may be configured to attach to the adhesive patch 106 in a way that allows the monitoring device 104 to receive signals from the light sensor 122 indicative of the reflected light sensed by the light sensor 122. In some embodiments, the monitoring device 104 includes the RF transmitter, RF receiver, at least one light source, and light sensor. For example, the RF transmitter and RF receiver may transmit and receive RF waves through the adhesive patch 106 on which the monitoring device 104 is mounted. Alternatively, in examples, the light source 120 and/or a light sensor 122 is disposed on a bottom surface of the monitoring device, and the adhesive patch 106 includes a transparent portion. As such, the light source 120 transmits light into the patient through the transparent portion, and the light sensor 122 may also receive reflected light through the transparent portion. In examples, the light source 120 may be disposed on a bottom surface of the monitoring device 104 and the light sensor 122 may be disposed in the adhesive patch. In examples, the light source 120 may be disposed in the adhesive patch and the light sensor 122 may be disposed on the bottom surface of the monitoring device 104.

In some embodiments, the monitoring device 104 and/or the adhesive patch 106 may include one or more additional sensors configured to sense other biometric signals of the patient. For instance, two or more ECG electrodes 114 may be embedded into the adhesive patch 106. In examples, the two or more ECG electrodes may be disposed on the bottom surface of the monitoring device 104. As such, the monitoring device 104 may receive signals from the ECG electrodes 114 indicative of the ECG of the patient. As another illustration, the monitoring device 104 may include a motion sensor configured to generate motion signals associated with the patient. Examples of this motion sensor may include a 1-axis channel accelerometer, 2-axis channel accelerometer, 3-axis channel accelerometer, multi-axis channel accelerometer, gyroscope, magnetometer, ballistocardiograph, and the like. In some embodiments, the portable gateway 108 may include one or more additional sensors configured to sense other biometric signals of the patient. For example, the portable gateway 108 may include a 3D accelerometer configured to generate motion signals associated with the patient.

The monitoring device 104 and adhesive patch 106 are configured for long-term and/or extended use or wear by, or attachment or connection to, a patient. For example, devices as described herein may be capable of being continuously used or continuously worn by, or attached to connected to, a patient without substantial interruption (e.g., 24 hours, 2 days, 5 days, 7 days, 2 weeks, 1 month, or beyond, such as multiple months, or even years). In some implementations, such devices may be removed for a period of time before use, wear, attachment, or connection to the patient is resumed. As an illustration, devices may be removed to change batteries, carry out technical service, update the device software or firmware, and/or take a shower or engage in other activities, without departing from the scope of the examples described herein. Such substantially or nearly continuous use, monitoring, or wear as described herein may nonetheless be considered continuous use, monitoring, or wear.

In some embodiments, the monitoring device 104 is configured to monitor, record, and transmit signals (e.g., RF sensor signals and light sensor signals) to the portable gateway 108 continuously. The monitoring device 104 monitoring and/or recording additional data may not interrupt transmitting already acquired data to the portable gateway 108. As such, in some embodiments, both the monitoring/recording and the transmission processes may occur at the same time or nearly the same time. In some embodiments, if the monitoring device 104 does suspend the monitoring and/or recording of additional data while it is transmitting already acquired data to the portable gateway 108, the monitoring device 104 may then resume monitoring and/or recording additional data prior to all of the already acquired data being transmitted to the portable gateway 108. To illustrate, the interruption period for monitoring and/or recording may be less in comparison to the time it takes the monitoring device 104 to transmit the already acquired data (e.g., between about 0% to about 80%, about 0% to about 60%, about 0% to about 40%, about 0% to about 20%, about 0% to about 10%, about 0% to about 5%, including values and subranges therebetween). This moderate interruption period may facilitate the near-continuous monitoring and/or recording of additional data during transmission of already acquired physiological data. For example, in one scenario, when a measurement time is around two minutes, any period of suspension or interruption in the monitoring and/or recording of subsequent measurement data may range from a few milliseconds to about a minute. Illustrative reasons for such suspension or interruption of data may include allowing for the completion of certain data integrity and/or other online test of previously acquired data. If the previous data has problems, the monitoring device 104 may notify the patient and/or a remote technician of the problems so that appropriate adjustments can be made.

In some embodiments, the monitoring device 104 may be configured to monitor, record, and transmit some data in a continuous or near-continuous manner as discussed above, while monitoring, recording, and transmitting some other data in a non-continuous manner (e.g., periodically, non-periodically, etc.). For example, the monitoring device 104 may be configured to record and transmit ECG data from the ECG electrodes 114 continuously or nearly continuously while data from the RF receiver is transmitted periodically (e.g., because RF measurements may be taken only when the patient is in a good position for transmitting and receiving RF signals, such as when the patient is not moving). As an illustration, ECG data may be transmitted to the portable gateway 108 (and, via the portable gateway 108, to the remote server 102) continuously or near-continuously as additional ECG data is being recorded, while RF sensor signals may be transmitted once the RF measuring process is completed. In some embodiments, monitoring and/or recording of signals by the monitoring device 104 may be periodic and, in some embodiments, may be accomplished as scheduled (e.g., periodically) without delay or latency during the transmission of already acquired data to the portable gateway 108. For example, the monitoring device 104 may sense signals or acquire signals from the patient in a periodic manner and transmit the data to the portable gateway 108 in a continuous manner as described above.

As discussed above, the portable gateway 108 is configured to receive the signals provided by the monitoring device 104 (e.g., RF sensor signals and light sensor signals) and transmit the signals to the remote server 102. Accordingly, the portable gateway 108 may be in wired and/or wireless communication with the monitoring device 104 and the remote server 102. As an illustration, the portable gateway 108 may communicate with the monitoring device 104 via Ethernet, via Wi-Fi, via RF, via near-field communication (NFC), and the like. The portable gateway 108 may further communicate with the remote server 102 via cellular networks, via Bluetooth®-to-TCP/IP access point communication, via Ethernet, via Wi-Fi, and the like. As such, the portable gateway 108 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the communications circuitry in the cardiac sensor and/or the portable gateway may communicate with the remote server over a Wi-Fi communications link based on the IEEE 802.11 standard. In some implementations, the cardiac sensor and/or portable gateway device may be part of an Internet of Things (IoT) and communicate with each other and/or the remote server 102 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

In some embodiments, the portable gateway 108 may continuously transmit the signals provided by the monitoring device 104 to the remote server 102. Thus, for example, the portable gateway 108 may transmit the signals from the monitoring device 104 to the remote server 102 with little or no delay or latency. To this end, in the context of data transmission between the cardiovascular monitoring unit 100, continuously includes continuous (e.g., without interruption) or near continuous (e.g., within one minute after completion of a measurement and/or an occurrence of an event on the monitoring device 104). Continuity may also be achieved by repetitive successive bursts of transmission (e.g., high-speed transmission). Similarly, immediate includes occurring or done immediately or nearly immediately (e.g., within one minute after the completion of a measurement and/or an occurrence of an event on the monitoring device 104).

Further, in the context of signal acquisition and transmission by the cardiovascular monitoring unit 100, continuously also includes uninterrupted collection of data sensed by the cardiovascular monitoring unit 100, such as RF sensor signals and light sensor signals, with clinical continuity. In this case, short interruptions in data acquisition of up to one second several times an hour, or longer interruptions of a few minutes several times a day may be tolerated, and still seen as continuous. As to latency as a result of such a continuous scheme as described herein, the overall amount of response time (e.g., time from when an event onset is detected to when a notification regarding the event is issued) can amount, for example, from about five to fifteen minutes. As such, transmission/acquisition latency may therefore be in the order of minutes.

In some embodiments, the bandwidth of the link between the monitoring device 104 and the portable gateway 108 may be larger, and in some instances, significantly larger than the bandwidth of the acquired data to be transmitted via the link (e.g., burst transmissions). Such embodiments may ameliorate issues that may arise during link interruptions, periods of reduced/absent reception, etc. In some embodiments, when transmission is resumed after the interruption, the resumption may be in the form of last-in-first-out (LIFO). Additionally, in some embodiments, the portable gateway 108 may be configured to operate in a store and forward mode where the data received from the monitoring device 104 is first stored in an onboard memory of the portable gateway 108 and then forwarded to the remote server 102. In some embodiments, the portable gateway 108 may function as a pipeline and pass through data from the monitoring device 104 immediately to the remote server 102. Further, in some embodiments, the data from the monitoring device 104 may be compressed using data compression techniques to reduce memory requirements as well as transmission times and power consumption.

Alternatively, in some embodiments, the monitoring device 104 may be configured to transmit the sensed or acquired signals to the remote server 102 instead of, or in addition to, transmitting the signals to the portable gateway 108. Accordingly, the monitoring device 104 may be in wired or wireless communication with the remote server 102. As an illustration, the monitoring device 104 may communicate with the remote server 102 via cellular networks, via Ethernet, via Wi-Fi channels, and the like. Further, in some embodiments, the cardiovascular monitoring unit 100 may not include the portable gateway 108. In such embodiments, the monitoring device 104 may perform the functions of the portable gateway 108 described above. Additionally, in such embodiments, the monitoring device 104 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the communications circuitry in the cardiac sensor and/or the portable gateway may communicate with the remote server over a Wi-Fi communications link based on the IEEE 802.11 standard. In some implementations, the cardiac sensor and/or portable gateway device may be part of an Internet of Things (IoT) and communicate with each other and/or the remote server 102 via IoT protocols for handling secure (e.g., encrypted) messaging and routing (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

The charger 110 includes charging cradles configured to hold and recharge the monitoring device 104 and the portable gateway 108. Alternatively, in some embodiments, the cardiovascular monitoring unit 100 may not include the portable gateway 108, and accordingly, the charger 110 may be configured to hold the monitoring device 104 alone.

The remote server 102 is configured to receive and process the signals transmitted by the monitoring device 104. Accordingly, the remote server 102 may include a computing device, or a network of computing devices, including at least one database (e.g., implemented in non-transitory media or memory) and at least one processor configured to execute instructions (e.g., stored in the database, with the at least one processor being in communication with the database) to receive and process the signals transmitted by the cardiovascular monitoring unit 100. In various embodiments, the remote server 102 identifies fiducial points on the aortic region and/or arterial waveforms represented by the RF sensor signals and light sensor signals. The remote server 102 then uses these fiducial points to determine one or more cardiovascular measures that indicate the health of the patient's cardiovascular system, as described in further detail below. Alternatively, in some embodiments, the monitoring device 104 may identify the fiducial points and/or determine the one or more cardiovascular measures and transmit this identified or determined information to the remote server 102 via the portable gateway 108.

As shown in FIG. 1, in some embodiments, the cardiovascular monitoring system further includes one or more user interfaces, such as technician interfaces 116 and caregiver interfaces 118. The technician interfaces 116 and caregiver interfaces 118 are in electronic communication with the remote server 102 through a wired or wireless connection. For instance, the technician interfaces 116 and caregiver interfaces 118 may communicate with the remote server 102 via Wi-Fi, via Ethernet, via cellular networks, and the like. Additionally, as shown, at least some of the technician interfaces 116 may also be in electronic communication with at least some of the caregiver interfaces 118 through a wired or wireless connection, such as via Wi-Fi, via Ethernet, via cellular networks, and the like. The technician interfaces 116 and the caregiver interfaces 118 may include, for example, desktop computers, laptop computers, and/or portable personal digital assistants (e.g., smartphones, tablet computers, etc.).

In some embodiments, the technician interfaces 116 are configured to electronically communicate with the remote server 102 for the purpose of viewing and analyzing information gathered from one or more monitoring devices 104 (e.g., aortic region and arterial waveforms, fiducial points on the aortic region and/or arterial waveforms, cardiovascular measures, etc.). For example, a technician interface 116 may provide one or more instructions to the remote server 102 to prepare a summary report of the cardiovascular measures for the patient for a certain time period. Accordingly, a technician interface 116 may include a computing device having a processor communicably connected to a memory and a visual display. The technician interface 116 may display to a user of the technician interface 116 (e.g., a technician) information gathered from the one or more monitoring device 104. The user of the technician interface 116 may then provide one or more inputs to the remote server 102 to guide the remote server 102 in preparing a report on the patient. As an example, a user may select a time period to use for a report, and the remote server 102 may prepare a report corresponding to the selected time period. As another example, a user may view a report prepared by the remote server 102 and draft a summary of the report that is included in a summary section of the report. As another example, a user may view waveforms provided by a monitoring device 104 and select fiducial points on those waveforms. The remote server 102 may then use the fiducial points input by the user to determine one or more cardiovascular measurements for the patient. Alternatively, in some embodiments, the remote server 102 may analyze and/or summarize the information gathered from one or more monitoring devices 104 with minimal or no input or interaction with a technician interface 116. In this way, the remote server 102 may analyze and/or summarize the information gathered from the one or more monitoring devices 104 through a completely or mostly automated process.

The caregiver interfaces 118 are configured to electronically communicate with the remote server 102 for the purpose of viewing information on patients wearing monitoring devices 104. As such, a caregiver interface 118 may include a computing device having a processor communicably connected to a memory and a visual display. The caregiver interface 118 may display to a user of the caregiver interface 118 (e.g., a physician, a nurse, or other caregiver) aortic region and/or arterial waveforms, fiducial points on the aortic region and/or arterial waveforms, cardiovascular measures, reports summarizing cardiovascular measurements, and/or the like for a patient. In some implementations, the user of the caregiver interface 118 may be able to interact with the displayed information on a patient wearing a monitoring device 104. For example, the user of the caregiver interface 118 may be able to select a portion of a patient report and, in response, be able to view additional information relating to the selected portion of the report, such as the aortic region and/or arterial waveforms used to generate the data included in the report. In some implementations, the user of the caregiver interface 118 may instead view a patient report without being able to interact with the patient report.

In some implementations, a technician interface 116 and/or a caregiver interface 118 may be a specialized user interface configured to communicate with the remote server 102. As an example, the technician interface 116 may be a specialized user interface configured to receive preliminary patient reports from the remote server 102, receive inputs from a user to adjust the preliminary report, and transmit the input to a remote server 102. The remote server 102 then uses the input from the technician interface 116 to prepare a finalized patient report, which the remote server 102 also transmits to the technician interface 116.

In some implementations, a technician interface 116 and/or a caregiver interface 118 may be a generalized user interface that has been adapted to communicate with the remote server 102. To illustrate, the technician interface 116 may be a user interface executing a technician application that configures a portable personal digital assistant to communicate with the remote server 102. For example, the technician application may be downloaded from an application store or otherwise installed on the user interface. Accordingly, when the user interface executes the technician application, the user interface is configured to communicate with the remote server 102 to receive and transmit information on patient wearing monitoring devices 104. Similarly, the caregiver interface 118 may be a user interface executing a caregiver application that configures the user interface to communicate with the remote server 102. The caregiver application may be similarly downloaded from an application store or otherwise installed on the user interface and, when executed, may be configure the user interface to communicate with the remote server 102 to receive and display information on patients wearing monitoring devices 104. The application store is typically included within an operating system of the device implementing the user interface. For example, in a device implementing an operating system provided by Apple Inc. (Cupertino, Calif.), the application store can be the App Store, a digital distribution platform, developed and maintained by Apple Inc., for mobile apps on its iOS and iPad OS operating systems. The application store allows user to browse and download a technician and/or caregiver interface app developed with an accordance with Apple iOS Software Development Kit. For example, such technician and/or caregiver interface apps can be downloaded on the iPhone smartphone, the iPod Touch handheld computer, or the iPad tablet computer, and some can be transferred to the Apple Watch smartwatch.

In some cases, the technician application and the caregiver application may be the same application, and the application may provide different functionalities to the device executing the application based on, for example, credentials provided by the user. For instance, the application may provide technician functionalities to a first user interface in response to authenticating technician credentials entered on the first user interface, and may provide caregiver functionalities to a second user interface in response to authenticating caregiver credentials entered on the second user interface. In other cases, the technician application and the caregiver application may be separate applications, each providing separate functionalities to a user device executing them.

In some implementations, the system shown in FIG. 1 may include other types of interfaces. To illustrate, in some examples, the system may include patient interfaces. Thus, the remote server 102 and/or a technician interface 116 may provide a report on a patient wearing a cardiovascular monitoring device 104 to the patient vi a patient interface. This patient report may be the same as a report provided to a caregiver via a caregiver interface 118, or this patient report may be different from the report provided to a caregiver via a caregiver interface 118. For instance, the report provided to a patient may be an abridged version of the patient report prepared for the caregiver. In various implementations, the patient interface may be configured similarly to and function similarly to the caregiver interface 118 discussed above (e.g., with some additional restrictions on what is included in a report and/or functionalities the patient can access).

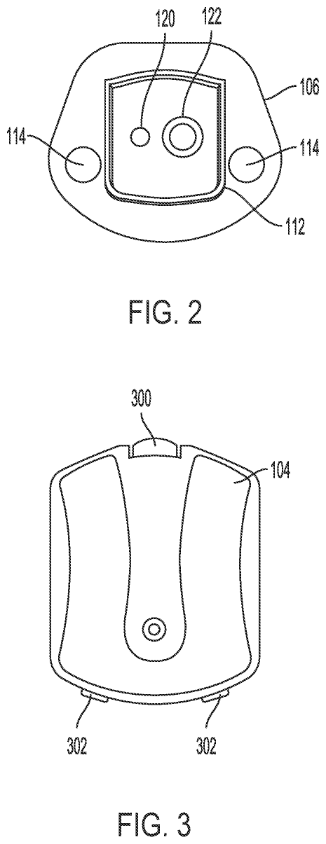

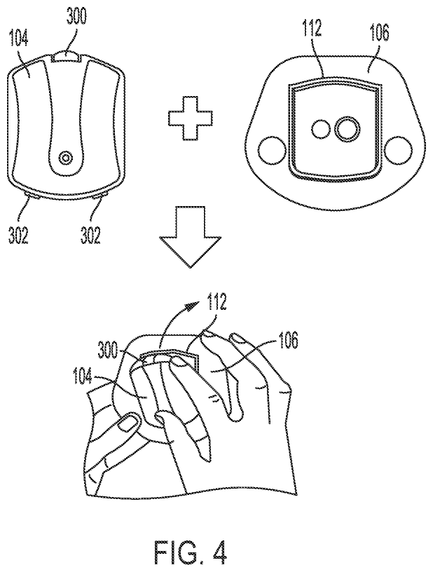

Returning to the monitoring device 104 and the adhesive patch 106, FIGS. 2-4 show the monitoring device 104 and the adhesive patch 106 according to some implementations. The adhesive patch 106 may be disposable (e.g., single- or few-use patches) and may of a biocompatible, non-woven material. Additionally, as shown in FIG. 2, and as noted above, the adhesive patch 106 may include a patch frame 112 delineating the boundary of the region of the patch 106 that is configured to house the monitoring device 104. In some embodiments, the monitoring device 104 may be designed for long-term usage. In such embodiments, the connection between the adhesive patch 106 and the monitoring device 104 may be configured to be reversible, e.g., the monitoring device 104 may be configured to be removably attached to the adhesive patch 106. For example, as shown in FIG. 3, the monitoring device 104 may include components such as snap-in clips 300 that are configured to secure the monitoring device 104 to the adhesive patch 106 upon attachment to the patch frame 112. After the monitoring device 104 is attached to the patch frame 112, a user may press the snap-in clip 300 to subsequently release the release the monitoring device 104 from the patch frame 112. The monitoring device 104 may also include positioning tabs 302 that facilitate the attachment process between the monitoring device 104 and the adhesive patch 106. For example, the positioning tabs 302 may guide a user to insert the monitoring device 104 onto the correct portion of the patch frame 112 such that the monitoring device 104 can then be coupled, connected, or snapped into the patch frame 112 using the snap-in clip 300, as shown in FIG. 4. In some embodiments, the adhesive patch 106 may be designed to maintain attachment to skin of a patient for several days (e.g., in a range from about 4 days to about 10 days, from about 3 days to about 5 days, from about 5 days to about 7 days, from about 7 days to about 10 days, from about 10 days to about 14 days, from about 14 days to about 30 days, etc.). After the period of use, the adhesive patch 106 may be removed from the patient's skin and the monitoring device 104 can be removed from the patch 106. The monitoring device 104 can be removably coupled, connected, or snapped onto a new adhesive patch 106 and reapplied to the patient's skin.

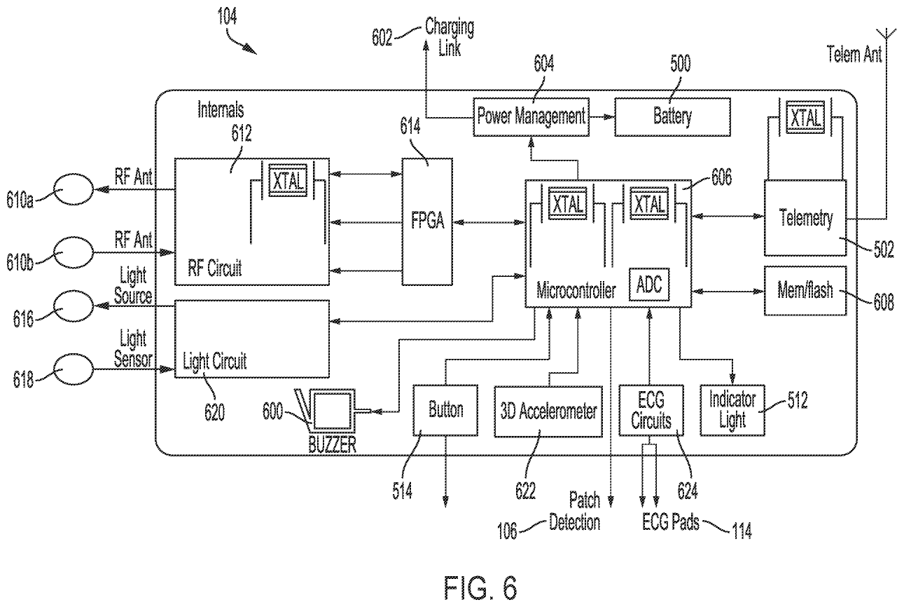

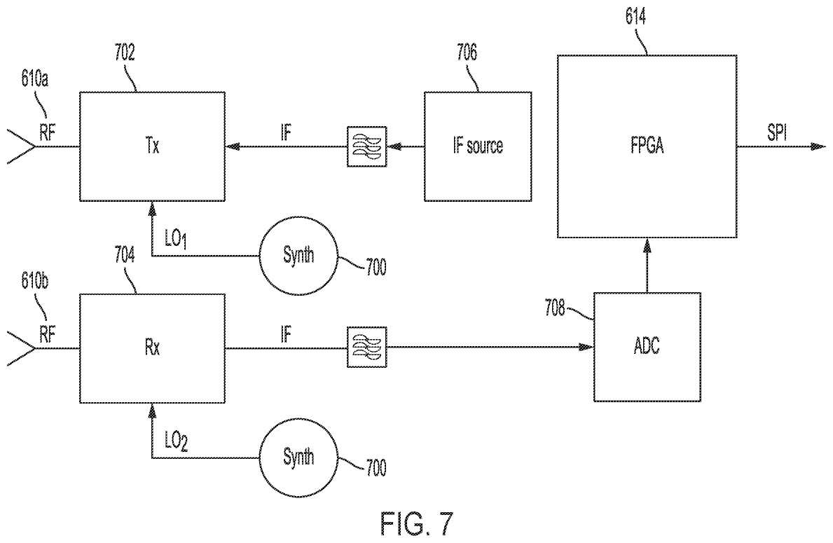

In some embodiments, the adhesive patch 106 includes additional components that facilitate or aid with the monitoring and/or recording or acquiring of RF sensor signals and light sensor signals by the monitoring device 104. For example, as shown in FIG. 2, the adhesive patch 106 may include one or more embedded light sources 120 configured to direct light into the patient's body and a light sensor 122 configured to detect light reflected from the patient's body. As such, the faces of the one or more light sources 120 and the light sensor 122 configured to generate and detect light, respectively, may be configured to contact the surface of the patient's skin (e.g., be positioned on the opposite side of the adhesive patch 106 from the side displayed in FIG. 2). The one or more light sources 120 and the light sensor 122 may be coupled to the monitoring device 104 by dedicated wiring within the adhesive patch 106. In some embodiments, the at least one light sensor and/or light source may be instead incorporated into the monitoring device 104, as described in further detail below. In some implementations, the adhesive patch 106 may include a printed circuit board (PCB) with some or all of the sensors (e.g., RF transmitter/receiver, light sensor, ECG sensor, etc.), circuitry, and antennae discussed herein. As such, the PCB of an adhesive patch 106 may include some of the functionality of the monitoring device 104 described below (e.g., with reference to FIGS. 6 and 7).

Additionally, in some embodiments, the adhesive patch 106 may include additional components that facilitate or aid with the monitoring and/or recording or acquiring of physiological data by the monitoring device 104. For instance, as discussed above, the adhesive patch 106 may include conductive elements such as one or more ECG electrodes 114 (e.g., a single lead, two leads, etc.) that can be used when recording ECG signals from the surface (e.g., skin contacted directly or through a covering) of a patient's body. The electrodes 114 may be coupled to the monitoring device 104 by dedicated wiring within the patch. In some embodiments, the ECG may have a sampling rate in the range from about 250 Hz to about 500 Hz, from about 300 Hz to about 450 Hz, from about 350 Hz to about 400 Hz, including values and subranges therebetween. In some embodiments, the ECG signal may be sampled after band-pass filtering by a 12-bit analog-to-digital converter (“ADC”). During normal operation, data may be transferred to the server “as-is” and can then be used by the remote server 102 for analysis. In some embodiments, an internal process allows for real-time evaluation of the ECG signal quality upon each attachment of the device to the patient.

In some embodiments, the remote server 102 and/or the monitoring device 104 may process the ECG signals to detect an arrhythmia of the patient. Types of arrhythmias detected by the remote server 102 and/or the monitoring device 104 may include ventricular ectopic beats (VEB), ventricular runs/ventricular tachycardia, bigeminy, supraventricular ectopic beats (SVEB), supraventricular tachycardia, atrial fibrillation, ventricular fibrillation, pauses, 2nd AV blocks, 3rd AV blocks, bradycardia, and/or other types of tachycardia. Additionally, the remote server 102 and/or the monitoring device 104 may perform other processing or analyses of the ECG signal, such as band pass filtering, detecting R-R intervals, detecting QRS intervals, and/or heart rate estimation.

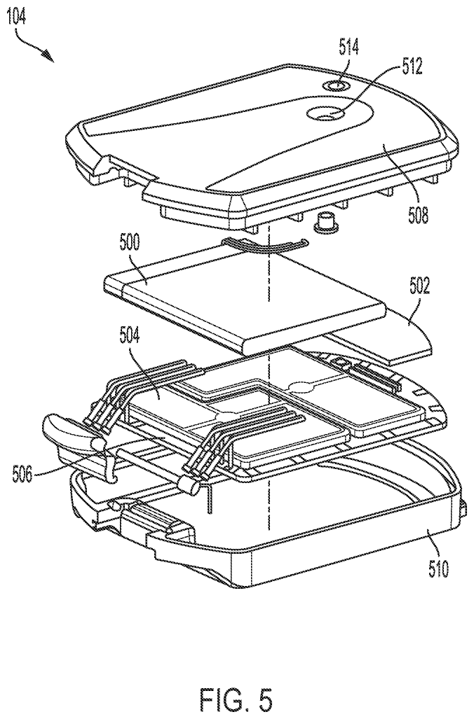

FIG. 5 provides an exploded view of the monitoring device 104, according to some embodiments. The exploded view of FIG. 5 illustrates various components of the monitoring device 104. For example, the monitoring device 104 may include a power source, such as a battery 500. In examples, the battery 500 may include a rechargeable lithium ion battery configured to supply power for at least one month of continuous or near-continuous RF, light, and/or ECG recording. The monitoring device 104 may also include a wireless communications circuit 502; a radio frequency shield 504 (e.g., a metallic cover, for instance, to prevent interferences with the RF and light processing and other digital circuitry); a digital circuit board 506; and/or the like. The wireless communications circuit 502 may be a Bluetooth® unit, in some embodiments, although in addition to or alternatively to the Bluetooth® unit, other modules facilitating other types of communications (e.g., Wi-Fi, cellular, etc.) may be included in the monitoring device 104.

These components may be provided between a front cover 508 forming an upper surface of the monitoring device 104 and a back cover 510 forming a bottom surface of the monitoring device 104 (e.g., with the back cover 510 configured to contact the adhesive patch 106 and the front cover 508 configured to face away from the patient such that the front cover is accessible when the monitoring device 104 is attached to the adhesive patch 106). In some embodiments, a light indicator 512 and/or a button 514 may be embedded into the front cover 508 visible through the upper surface. The light indicator 512 may provide feedback on the status of the monitoring device 104 and its components, such as the charging and/or power level of the power source of the monitoring device 104 (e.g., the battery 500), the attachment level of the monitoring device 104 to the adhesive patch 106, the attachment level of the adhesive patch 106 to the surface of the body to which the adhesive patch 106 is attached, etc. The button 514 may be configured for the patient and/or a caregiver to provide feedback to the monitoring device 104 and/or the remote server 102. For instance, the button 514 may allow the patient and/or a caregiver to activate or deactivate the monitoring device 104. In some implementations, the button 514 may be used to reset the monitoring device 104, as well as pair the monitoring device 104 to the portable gateway 108 and initiate communication with the portable gateway 108. In some embodiments, the button 514 may allow a user to set the monitoring device 104 in an “airplane mode,” for example, by deactivating any wireless communication (e.g., Wi-Fi, Bluetooth®, etc.) with external devices and/or servers, such as the portable gateway 108 and/or the remote server 102.

FIG. 6 illustrates an example electronic architecture for the monitoring device 104. In some embodiments, as shown in FIG. 6, the monitoring device 104 includes one or more external interfaces, either connected to or embedded in the monitoring device 104. For example, the monitoring device 104 may include the button or switch 514 for activating the monitoring device 104, deactivating the monitoring device 104, pairing the monitoring device 104 with the portable gateway 108, receiving patient input, and/or the like. In some embodiments, the monitoring device 104 may also include the light indicator 512 and a buzzer 600 for providing audio feedback to a user of the monitoring device 104 (e.g., in response to the patient activating the button 514 or tapping the monitoring device 104 to record that the patient is experiencing symptoms suspected to be related to an arrhythmia). Further, in some embodiments, the monitoring device 104 may be connectable to the ECG pads or electrodes 114 coupled to the patient (e.g., connectable to the ECG pads 114 embedded in the adhesive patch 106) and to a charger, such as the charger 110, via a charging link 602. For instance, the back cover 510 of the monitoring device 104 may include metal contacts configured to connect to the ECG pads 114 when the monitoring device 104 is attached to the adhesive patch 106 and to a charging power source when the monitoring device 104 is attached to the charger 110. The ECG circuits 624 may receive signals from the ECG pads 114 when the monitoring unit 104 is attached to the adhesive patch 106, where the signals received from the ECG pads 114 include ECG waveforms sensed from the patient. it Alternatively, or additionally, in some embodiments, the monitoring device 104 may include an inductive circuit configured to charge the monitoring device 104 via a wireless inductive charging link 602. As shown in FIG. 6, the charging link 602 may be coupled to a power management circuit 604 (e.g., when the monitoring device 104 is attached to the charger 110, when the monitoring device 104 is placed in proximity to an inductive charging pad), where the power management circuit 604 is configured to charge an onboard power source, such as the battery 500.

Internally, in some embodiments, the monitoring device 104 may include a microprocessor (e.g., being connected to a separate non-volatile memory, such as memory 608) or a microcontroller 606. The microcontroller 606 stores instructions specifying how measurements (e.g., RF, light, ECG, accelerometer, etc.) are taken, how obtained data are transmitted, how to relay a status of the monitoring device 104, how/when the monitoring device 104 can enter a sleep level, and/or the like. In some embodiments, the instructions may also specify the conditions for performing certain types of measurements. For example, the instructions may specify that an accelerometer of the monitoring device 104 may not commence measurements (e.g., for RF data, light data, ECG data, etc.) unless the patient using the monitoring device 104 is at rest or maintaining a certain posture. As another example, the instructions may identify the conditions that may have to be fulfilled before measurements can commence, such as a sufficient attachment level between the monitoring device 104 and the adhesive patch 106 and/or a sufficient attachment level between the adhesive patch 106 and the surface of the body onto which the adhesive patch 106 is attached. In some embodiments, the microcontroller 606 may have internal and/or external non-volatile memory banks (e.g., memory 608) that can be used for keeping measurement directories and data, scheduler information, and/or a log of actions and errors. This non-volatile memory allows saving power via a total power down while retaining data and status information.

As discussed above, in various embodiments, the monitoring device 104 includes RF antennae for directing electromagnetic waves into a body of a patient and receiving waves that are scattered and/or reflected from internal tissues. The RF antennae may be flat, printed, set flush against the skin, with or without an interface material, and/or the like. The RF antennae may be in a bow-tie, spiral, monostatic, bistatic, and/or like configurations. Further, the monitoring device 104 includes RF circuitry configured to process the received waves so as to determine some properties of the tissues that are on the path of the transmitted and/or scattered/reflected waves. For example, the antennae may direct RF waves towards an aortic region of a patient. The RF circuitry may receive scattered/reflected waves from the aortic region and generate RF sensor signals that include information about an aortic region waveform of the patient.

As such, FIG. 7 shows an example embodiment of the monitoring device 104 including RF antennae 610a, 610b, an RF circuit 612, and other circuits for controlling the RF circuit (e.g., field-programmable gate array (FPGA) circuits 614). In various embodiments, the RF antennae 610a, 610b are configured to transmit RF waves to the body of a patient to which the monitoring device 104 is attached and receive scattered/reflected RF waves from the body of the patient. FIG. 7 illustrates block diagrams that illustrate examples of RF sensor functionality implemented within the RF circuit 612. Such functionality may be used to monitor the volume of the arteries in the patient's aortic region over time in accordance with the techniques described herein. As shown in FIG. 7, initially, one or more RF signals (e.g., a single local oscillator (LO) signal, or different “LO₁” and “LO₂” signals, collectively “LO signals”) can be generated by a broadband synthesizer 700 (e.g., a pulse generator and synthesizer, or local oscillator). Such a synthesizer 700 may include moderate phase noise performance and/or fast settling time capabilities. The RF circuit 612 also includes a transmitter portion 702, coupled to a transmitter RF antenna 610a (e.g., Tx) and associated circuitry for transmitting RF waves directed, for example, towards the patient's aortic region. The RF circuit 612 further includes a receiver portion 704 coupled to a receiver RF antenna 612b (e.g., Rx) and associated circuitry 482 for receiving reflected RF waves from, for example, the patient's aortic region.

In some embodiments, the LO signal of the transmitter portion 702 is multiplied with an external sine wave at a low frequency intermediate frequency (IF) signal, generated by an IF source 706, and directed to the output of the transmitter portion 702. As noted above, the LO signal at the transmitter portion 702 and the receiver portion 704 can be generated by one or more LOS sources (e.g., synthesizer(s) 700). Output power may be controlled via a digitally controlled attenuator (DCA) on the LO signal transmitter path. An external, reflected RF wave returning to the receiver RF antenna 610b may be directed to the receiver portion 704 and down-converted to an IF frequency by a down conversion mixer. The reflection characteristics (e.g., phase and amplitude) can be transformed to a new IF carrier (e.g., on the order of 250 kHz), filtered, and amplified, before being forwarded to an analog-to-digital converter (ADC) 708. In some embodiments, digital control for the functionality described with respect to FIG. 7 may be achieved directly by a processor and/or digital logic (e.g., an FPGA 614), which may be configured to control the transmitter and receiver configuration processes, IF signal adjustments, and associated switching. As shown in FIG. 7, the output of the RF circuit 612 may be in the form of serial peripheral interface (SPI).

Referring back to FIG. 6, as discussed above, the monitoring device 104 includes or is coupled to (e.g., when connected to the adhesive patch 106) at least one light source 616 for directing light waves of a predetermined frequency into the body of a patient and at least one light sensor 618 for receiving waves that are scattered and/or reflected from internal tissues. In some implementations, the at least one light source 616 may be at least one diode, such as at least one LED (e.g., a green LED, a red LED). In some implementations, the monitoring device 104 may include or be coupled to multiple light sources 616, where each source emits light of a different predetermined frequency. As an example, the monitoring device 104 may include or be coupled to a green LED and a red LED. In some implementations, the at least one light source 616 and/or the light sensor 618 may be external to the monitoring device 104. For instance, the at least one light source 616 and/or the light sensor 618 may be embedded in the adhesive patch 106 (e.g., as light source 120 and light sensor 122, shown in FIGS. 1 and 2) and connected to the monitoring device 104 via internal wiring of the adhesive patch 106. In some implementations, the at least one light source 616 and/or the light sensor 618 may be included in the monitoring device 104, such as placed on a bottom surface of the monitoring device 104, as described in further detail below.

Additionally, the monitoring device 104 includes light circuitry configured to process the received waves so as to determine some properties of the tissues that are on the path of the transmitted and/or scattered/reflected waves. For example, the light source 616 may direct light waves into the arteries near the skin surface of a patient, such as arteries above the sternum of the patient. The light sensor 618 may receive scattered light that has been reflected off of the sternum through the surface arteries. As such, the light circuit 620 may receive signals from the light sensor 618 and generate light sensor signals that include information about an arterial waveform of the patient.

In some embodiments, the monitoring device 104 may also include or be connected to one or more additional sensors. For example, as shown in FIG. 6, the monitoring device 104 may include a motion sensor such as a 3D accelerometer 622. Using the 3D accelerometer 622, the monitoring device 104 may acquire data on patient movements, patient orientation, patient respiration, and/or the like. The monitoring device 104 and/or the remote server 102 may use the acquired accelerometer data to determine physiological and/or biometric information for the patient, such as the patient's posture or orientation, activity rate, respiration rate, and/or the like. In some implementations, the monitoring device 104 may use the physiological and/or biometric information, for example, to determine when to take RF and/or light measurements from the patient. As an illustration, to reduce artifacts and other bad readings, the monitoring device 104 may only or primarily take RF measurements from the patient when the monitoring device 104 determines from the accelerometer data that the patient is substantially stationary or otherwise inactive. For example, the monitoring device 104 can determine from the accelerometer data (e.g., accelerometer counts) that the patient motion is below a preset threshold to determine that the patient is substantially stationary or otherwise inactive.

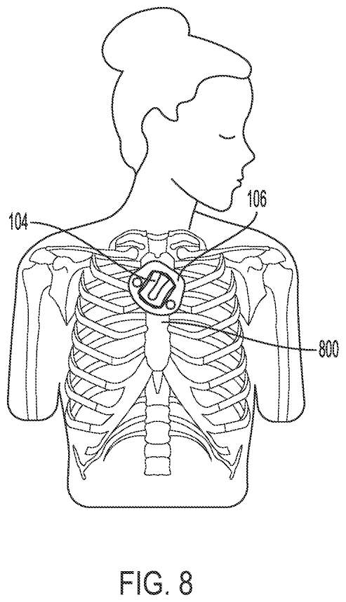

As discussed above, in some implementations, the cardiovascular monitoring unit 100 may include a monitoring device 104 that includes an RF transmitter and receiver, where the monitoring device 104 is configured to attach to an adhesive patch 106 that includes one or more light sources 120 and a light sensor 122, as shown in FIGS. 1-4. In such embodiments, the adhesive patch 106 is configured to be placed on a first location on a patient, with the monitoring device 104 configured to be attached to the adhesive patch 106 once placed. As an illustration, referring to FIG. 8, the adhesive patch 106 may be adhered to the patient's skin on the patient's thorax over the patient's sternum 800. Once the adhesive patch 106 is attached to the skin over the sternum 800, the patient or a caregiver may attach the monitoring device 104 to the adhesive patch 106 such that the adhesive patch 106 and the monitoring device 104 are positioned as shown in FIG. 8.

For example, as illustrated in FIG. 8, the adhesive patch 106 and monitoring device 104 may be positioned over the upper third of the patient's sternum 800, roughly level with the patient's aortic region (e.g., between the second and third costal notches). This placement may facilitate the monitoring device 104 in transmitting RF waves to and receiving scattered/reflected RF waves from the patient's aortic region. At the same time, this placement may also facilitate the one or more light sources (e.g., light source 120) embedded into the adhesive patch 106 in directing light to the surface arteries above the sternum 800 and receiving light reflected off the sternum at the light sensor embedded into the adhesive patch 106 (e.g., light sensor 122). However, the monitoring device 104 and the adhesive patch 106 may be placed on a different physiological location on the patient, in some implementations. For instance, the monitoring device 104 and the adhesive patch 106 may be placed on a lower third of the sternum 800 (e.g., to avoid being placed on top of a patient's breasts).

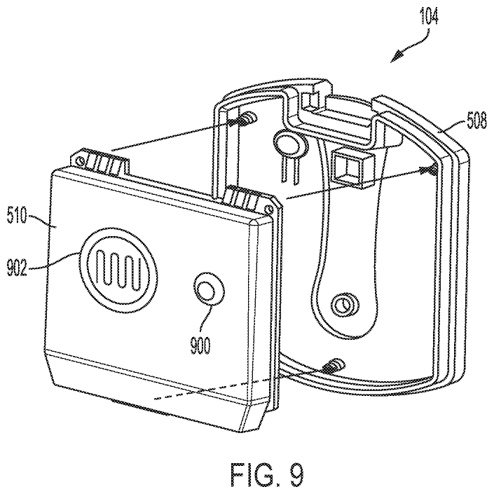

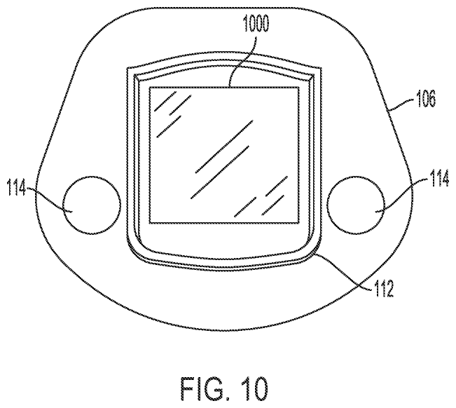

In some implementations, the adhesive patch 106 may not include the embedded one or more light sources and light sensor. Instead, as illustrated in FIG. 9, the monitoring device 104 may include one or more light sources 900 and a light sensor 902 in some implementations. For example, as shown, the light source 900 and the light sensor 902 may be mounted into the back cover 510 of the monitoring device 104. To allow the light source 900 to direct light into the arteries below the skin of the patient's thorax, and to allow the light sensor 902 to receive light reflected from the one or more arteries below the skin, at least a portion 1000 of the adhesive patch 106 may be transparent, as shown in FIG. 10. The transparent portion 1000 of the adhesive patch is configured to be below the light source 900 and the light sensor 902 of the monitoring device 104 once the monitoring device 104 is attached to the adhesive patch 106. As such, the light source 900 can direct light through the transparent portion 1000 and into the skin of the patient's thorax, and the light sensor 902 can also receive light through the transparent portion 1000 that is reflected from the arteries under the skin of the patient's thorax. In such implementations, the adhesive patch 106 and the monitoring device 104 may be positioned on the patient similarly to the positioning shown in FIG. 8 (e.g., over an upper third of the patient's sternum 800). Alternatively, in some implementations, the adhesive patch 106 may not include a transparent portion 1000 and may instead include one or more holes where the light source 900 and the light sensor 902 sit on the patch 106 when the monitoring device 104 is attached to the patch 106. As such, the light source 900 may transmit light through a hole and into the skin on the patient's thorax, and the light sensor 902 may receive reflected light through a hole.

In some implementations, the one or more light sources and the light sensor may be split between the monitoring device 104 and the adhesive patch 106. For example, the monitoring device 104 may include the light source 900 embedded on the back surface of the monitoring device 104. As such, the adhesive patch 106 may include the transparent portion 1000 such that the light source 900 can transmit light into the skin of the patient's thorax. The adhesive patch 106 may also include the light sensor 122, which senses reflected light from the patient's thorax and transmits signals indicative of the sensed light to the monitoring device 104 via internal wires of the adhesive patch 106.

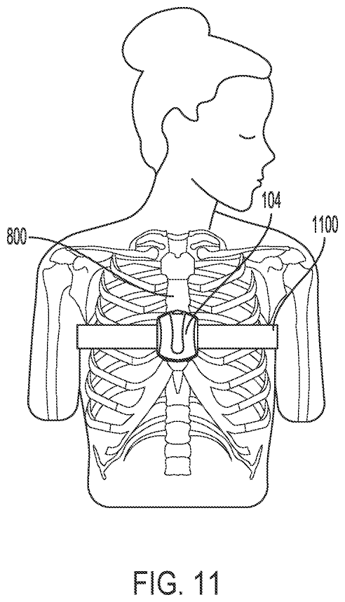

In some implementations, the cardiovascular monitoring unit 100 may not include an adhesive patch 106. Instead, the monitoring device 104 may be attached to the patient's body through another mechanical implement. As an illustration, as shown in FIG. 11, the cardiovascular monitoring unit 100 may include a band 1100 configured to encircle the patient's chest. The band 1100 may be made of an elastic material that compresses the band 1100 around the patient's chest to ensure a secure or relatively secure fit where the band 1100 does not slip on the patient's chest as the patient moves. The monitoring device 104 may be configured to be mounted onto the band 1100, as illustrated in FIG. 11. For example, the band 1100 may include a plastic frame, similar to the patch frame 112 of the adhesive patch 106 shown in FIGS. 1, 2, and 4, that the monitoring device 104 removably attaches onto. As another example, the band 1100 may include strips of hook or loop cloth configured to removably attach to matching strips of loop or hook cloth on the monitoring device 104.

In implementations where the monitoring device 104 is mounted onto the band 1100, the RF transmitter and receiver may be included in the monitoring device 104, as described above with respect to FIGS. 6 and 7. The one or more light sources and/or the light sensor may be included in the monitoring device 104 or the band 1100. For instance, a light source and a light sensor may be mounted into the inside of the band 1100 such that the light source and the light sensor contact the skin of the patient when the patient is wearing the band 1100 as shown in FIG. 11. As such, the light source can transmit light into the patient's thorax (e.g., above the patient's sternum 800 as shown in FIG. 11), and the light sensor can receive scattered/reflected light from the patient's thorax (e.g., scattered/reflected off of the sternum 800 and through the arteries above the patient's thorax). As another example, the monitoring device 104 may include a light source 900 and a light sensor 902 as shown in FIG. 9. A transparent patch, such as a transparent vinyl patch, may be constructed into the band 1100 where monitoring device 104 is mounted onto the band 1100 such that the light source 900 and light sensor 902 can transmit and receive light through the transparent patch. Alternatively, one or more holes may be constructed into the band 1100 such that the light source 900 and the light sensor 902 can transmit and receive light through the holes. As another example, the monitoring device 104 may include a light source and a light sensor on a bottom surface of the monitoring device 104 where the bottom surface contacts the skin instead of the band 1100. To illustrate, referring to FIG. 11, the monitoring device 104 may include a light source and a light sensor on the top third and/or the bottom third of the bottom surface of the monitoring device 104, as the middle third of the monitoring device 104 is the portion of the monitoring device 104 contacting the band 1100. The top third and/or bottom third of the bottom surface of the monitoring device 104 may directly contact or nearly contact the thorax of the patient, particularly if the band 1100 is made of a thin material. Accordingly, one or more light sources and a light sensor provided on the top third and/or bottom third of the monitoring device 104 may direct light into the patient's thorax and receive reflected light from the patient's thorax.

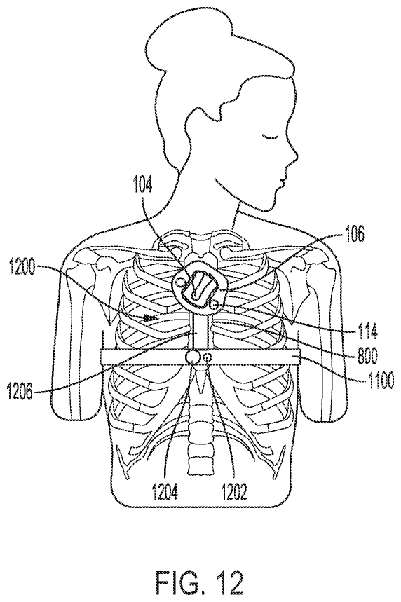

In some implementations, the cardiovascular monitoring unit 100 may include a combination piece for mounting the monitoring device 104, as well as the RF transmitter, RF receiver, one or more light sources, and light sensor, onto the patient's body. For example, FIG. 12 illustrates a wearable combination piece 1200 that includes an adhesive patch 106 and a band 1100. The adhesive patch 106 is configured to be adhered on a first location on the patient, such as above the patient's sternum, similar to the embodiment described with respect to FIG. 8. The band 1100 may be worn lower on the patient's thorax, such as over a lower portion of the patient's sternum 800 as shown in FIG. 12. As illustrated in the example embodiment of FIG. 12, in some implementations, one or more light sources 1202 and a light sensor 1204 may be affixed to the band 1100 where the band 1100 sits over the patient's sternum 800. An RF transmitter and RF receiver may be incorporated into the monitoring device 104 as described above with respect to FIGS. 6 and 7.

Additionally, this configuration for the wearable combination piece 1200 may include a connector 1206 configured to connect the adhesive patch 106 to the band 1100. The connector 1206 may be an extension of the adhesive patch 106, a piece of fabric, a piece of plastic, and/or the like. In some implementations, the connector 1206 may removably attach to each of the patch 106 and the band 1100 (e.g., through snaps, through hooks, through hook-and-loop fasteners, and/or the like). In some implementations, the connector 1206 may removably attach to one of the patch 106 and the band 1100. For example, the connector 1206 may be an extension of the adhesive patch 106, and the band 1100 may attach to the bottom portion of the connector 1206. In some implementations, the wearable combination piece 1200 may be formed as a single unit.

The connector 1206 may house one or more electrical components, such as wiring connecting the light source 1202 and the light sensor 1204 to the monitoring device 104. In some cases, the connector 1206 may also help ensure the correct placement of the wearable combination piece 1200 on the patient's body. For example, by restricting how the wearable combination piece 1200 can be worn, the connector 1206 may help ensure that the patch 106 and the band 1100 are both placed over the patient's sternum 800. Further, in some cases, the configuration of the wearable combination piece 1200 may help the wearable combination piece 1200 conform to the patient's body. As an example, the configuration of the wearable combination piece 1200 may allow the cardiovascular monitoring unit 100 to be more easily used by female patients, as the adhesive patch 106 may be worn above the patient's breasts and the band 1100 may be worn below the patient's breasts where a patch would be difficult to adhere above the patient's sternum. Additionally, moving the light source 1202 and the light sensor 1204 to the band 1100 may, in some implementations, allow the size of the monitoring device 104 to be decreased and thus the patch 106 size to be decreased. Decreasing the size of the monitoring device 104 and/or the patch 106 may further facilitate the placement of the wearable combination piece 1200 on female patients, where a larger monitoring device 104 and/or patch 106 may be difficult to place on female patients with larger breasts. In some implementations, ECG electrodes 114 may also be moved down to the band 1100 to further decrease the size of the adhesive patch 106.

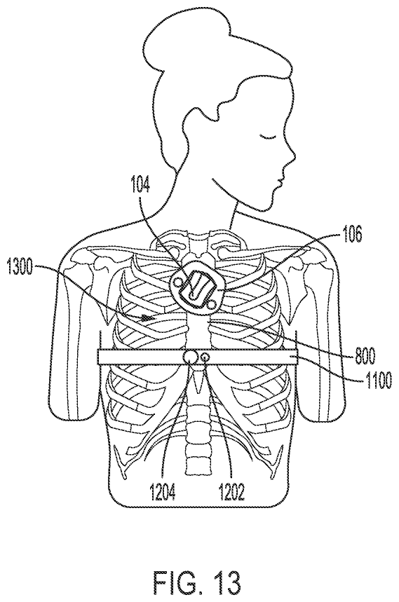

In some implementations, a combination piece for mounting the monitoring device 104 and the RF transmitter, RF receiver, one or more light sources, and light sensor onto the patient may be implemented as multiple, unconnected pieces. For example, as shown in FIG. 13, a wearable combination piece 1300 may include an adhesive patch 106 and a band 1100. The adhesive patch 106 and band 1100 may be implemented similarly to the embodiment shown in FIG. 12, including the band having the one or more light sources 1202 and light sensor 1204. However, unlike the wearable combination piece 1200, the adhesive patch 106 and the band 1100 in the wearable combination piece 1300 are not connected. As such, the light source 1202 and light sensor 1204 on the band 1100 may each have an independent power source (not shown). Additionally, the monitoring device 104 may be configured to wirelessly communicate with the light source 1202 and the light sensor 1204, for example, to control and receive measurements taken with the light source 1202 and the light sensor 1204.

In some implementations, the band 1100 may include a processor and a memory storing instructions and/or configured to receive instructions from the monitoring device 104 for controlling operation of the light source 1202 and the light sensor 1204. Additionally, the band 1100 may include communications circuitry for communicating with the monitoring device 104 and/or the portable gateway 108. In some implementations, the band 1100 may communicate directly with the monitoring device 104 (e.g., via Bluetooth®, via Wi-Fi, via radiofrequency communication (RFC), via near field communication (NFC), etc.). In some implementations, the band 1100 may communicate indirectly with the monitoring device 104, such as through the portable gateway 108. For example, in some implementations, the monitoring device 104 may transmit instructions for the light source 1202 and light sensor 1204 to take one or more measurements from the patient to the portable gateway 108. The portable gateway 108 may then transmit the instructions to the band 1100. The light source 1202 and light sensor 1204 may take the measurements and transmit the measurements, via the communications circuitry of the band 1100, to the portable gateway 108. The portable gateway 108 may transmit the measurements to the monitoring device 104 or, alternatively or additionally, directly to the remote server 102.

In some cases, the implementation of the wearable combination piece 1300 as a separate adhesive patch 106 and band 1100 may allow the patient to wear the adhesive patch 106 and the monitoring device 104 continuously or nearly continuously but remove the band 1100 when the light source 1202 and light sensor 1204 are not being used. As such, the embodiment of the wearable combination piece 1300 shown in FIG. 13 may include the benefits of the embodiment of the wearable combination piece 1200 shown in FIG. 12, as well as further providing for the patient's comfort by making the band 1100 removable.

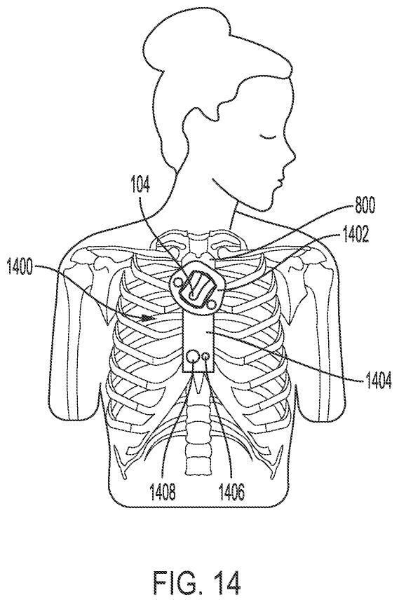

In some implementations, the cardiovascular monitoring unit 100 may include an adhesive patch with a different configuration from the adhesive patch 106 shown in FIGS. 8, 10, 12, and 13. For example, the cardiovascular monitoring unit 100 may include an adhesive patch 1400 configured to cover a larger area of the patient's sternum, as shown in FIG. 14. The adhesive patch 1400 may include a top portion 1402 configured to be placed over an upper part of the patient's sternum 800 and a bottom portion 1404 that extends down the patient's sternum 800. The top portion 1402 is also configured to receive the monitoring device 104, as shown in FIG. 14. The RF transmitter and RF receiver may be incorporated into the monitoring device 104, as described above with reference to FIGS. 6-7, and one or more light sources 1406 and a light sensor 1408 may be set into the bottom portion 1404 over the lower part of the patient's sternum 800. For example, the light source 1406 and light sensor 1408 may be removably set into the bottom portion 1404 of the adhesive patch 1400 (e.g., the light source 1406 and light sensor 1408 may couple, connect, or snap into the adhesive patch 1400) or may be permanently set into the bottom portion 1404 of the adhesive patch 1400. The adhesive patch 1400 may include internal wiring that facilitates communication between the monitoring device 104 and the light source 1406 and light sensor 1408.

In some cases, the adhesive patch 1400 may have configurations of different lengths and/or different placements of the light source 1406 and the light sensor 1408. As such, a caregiver may be able to select an adhesive patch 1400 that helps ensure the light source(s) 1406 and light sensor 1408 are placed over the section of the sternum 800 that provides for the best light measurements from the patient.

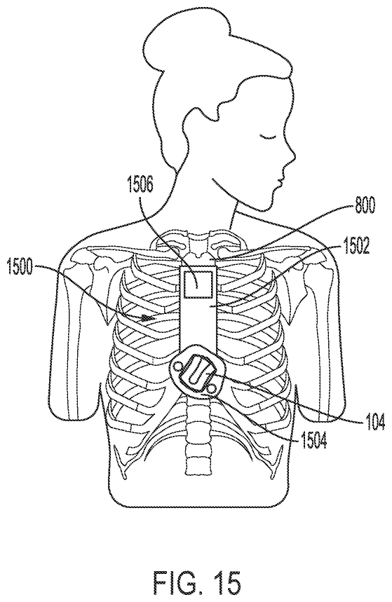

Another implementation for an adhesive patch 1500 is shown in FIG. 15. As illustrated, the adhesive patch 1500 includes a top portion 1502 configured to be placed over an upper and middle section of the patient's sternum 800. The adhesive patch 1500 also include a bottom portion 1504 configured to be placed over a lower part of the patient's sternum 800, as shown. The top portion 1502 is configured to receive an RF unit 1506 that includes an RF transmitter, RF receiver, associated circuitry (e.g., similar to the RF circuitry shown in FIG. 7), and a power source. For example, the RF unit 1506 may be removably attached to the adhesive patch 1500 via a frame on the top portion 1502 of the adhesive patch 1500 (e.g., similar to the patch frame 112 of the adhesive patch 106 described above). The bottom portion 1504 is configured to receive the monitoring device 104, as shown in FIG. 15. Additionally, the monitoring device 104 and/or the bottom portion 1504 of the adhesive patch 1500 may include one or more light sources and a light sensor, similar to the embodiments of the monitoring device 104 and adhesive patch 106 described above with reference to FIGS. 2 and 9-10.

In some cases, the adhesive patch 1500 may have configurations of different lengths and/or different placements for the RF unit 1506, similar to the adhesive patch 1400 described above. A caregiver may thus be able to select an adhesive patch 1500 that helps ensure the RF unit 1506 and the light source(s) and light sensor are placed over the sternum 800 in such a way that provides the best RF and light measurements for the patient. Additionally, as shown in FIG. 15, this configuration of the adhesive patch 1500 may allow the monitoring device 104 to be placed lower on the patient's sternum 800. The ability to place the monitoring device 104 lower on the sternum may be beneficial, for example, for female patients who have breasts that make the placement of the monitoring device 104 and/or the adhesive patch or portion of adhesive patch that receives the monitoring device 104 higher on their thorax difficult.

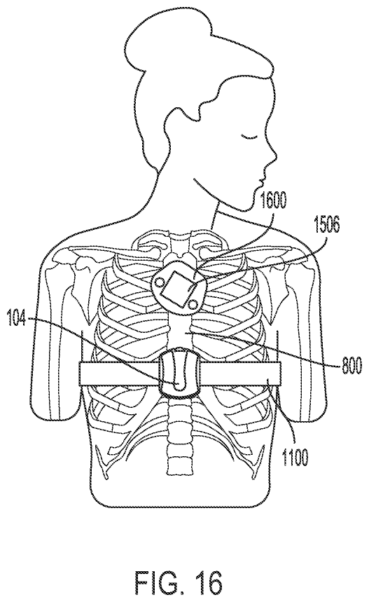

In some implementations, the placement of an RF unit higher on a patient's sternum and the monitoring device 104 lower on the patient's sternum may be facilitated by a wearable combination piece. As an example, FIG. 16 illustrates a patient with an adhesive patch 1600 placed over an upper portion of the patient's sternum 800 and a band 1100 placed over a lower portion of the patient's sternum 800. As shown, the adhesive patch 1600 may be configured similarly to the adhesive patch 106 discussed above with respect to FIGS. 2, 4, and 8 configured to receive the RF unit 1506 (e.g., via a frame similar to the patch frame 112 of the adhesive patch 106). The band 1100 is configured to receive the monitoring device 104 (e.g., as described with respect to FIG. 11). Similar to the embodiment shown in FIG. 13, the RF unit 1506 and the monitoring device 104 may communicate wirelessly, either directly or via the portable gateway 108.

FIG. 16 illustrates an example embodiment of a wearable combination piece (as well as FIGS. 12 and 13). Other configurations of pieces for mounting the components used to take RF and light measurements from the patient may be used. For instance, a patient may wear two adhesive patches, one over the upper portion of a first location on the patient (e.g., skin over an upper portion of the patient's sternum) and one over the lower portion of the first location on the patient (e.g., skin over a lower portion of the patient's sternum). An RF unit may be attached to the upper adhesive patch, and a monitoring device may be attached to the lower adhesive patch. In this way, the patient may only need to wear the adhesive patch or patches associated with the unit or device actually being used.

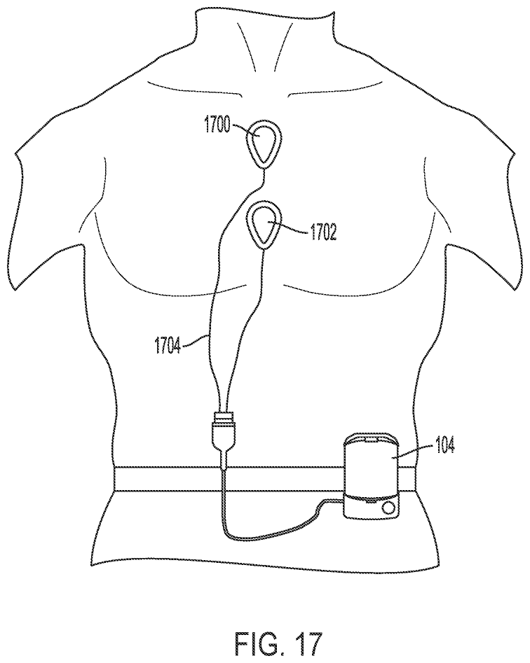

In some implementations, the monitoring device 104 may not be directly worn on the thorax of the patient. Instead, the components used to take RF and light measurements from the patient (e.g., the RF transmitter, RF receiver, light source, and light sensor) may be worn on the thorax of the patient and transmit RF and light data to the monitoring device 104 worn elsewhere on the patient. For example, FIG. 17 illustrates the monitoring device 104 being worn on a belt of the patient (e.g., via a belt clip, not shown). The monitoring device 104 is in wired communication with a first sensor patch 1700 and a second sensor patch 1702 via cables 1704. The first sensor patch 1700, which is shown as being placed higher on the patient's thorax, may include an RF transmitter and RF receiver (e.g., controlled and powered by the monitoring device 104). The second sensor patch 1702, which is shown as being placed lower on the patient's thorax, may include one or more light sources and a light sensor (e.g., controlled and powered by the monitoring device 104). In some cases, for example, the implementation shown in FIG. 17 may allow the cardiovascular monitoring unit 100 to be used by an individual who finds it difficult or uncomfortable to wear the monitoring device 104 on their thorax. Additionally, in some implementations, the first sensor patch 1700 and the second sensor patch 1702 may include one or more ECG electrodes. For example, a first ECG electrode may be embedded into the first sensor patch 1700, and a second ECG electrode may be embedded into the second sensor patch 1702. Thus, the monitoring device 104 may be able to generate ECG signals that include information about the patient's ECG based on electrical activity of the heart sensed between the ECG electrodes on the sensor patches 1700 and 1702.

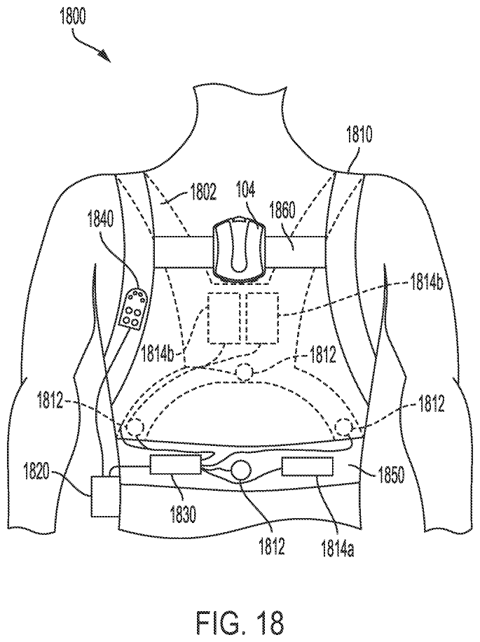

FIG. 18 shows another embodiment of the cardiovascular monitoring unit 100, where the cardiovascular monitoring unit 100 includes a garment-based medical device 1800. The garment-based medical device 1800 shown in FIG. 18 is external, ambulatory, and wearable by a patient 1802. Such a garment-based medical device 1800 can be, for example, an ambulatory medical device that is capable of and designed for moving with the patient 1802 as the patient goes about his or her daily routine. For example, the garment-based medical device 1800 as described herein can be bodily-attached to the patient 1802 such as the LifeVest® wearable cardioverter defibrillator available from ZOLL® Medical Corporation of Chelmsford, Mass. In one example scenario, such wearable defibrillators can be worn nearly continuously or substantially continuously for a week, two weeks, a month, or two or three months at a time. During the period of time in which they are worn by the patient 1802, the wearable defibrillators can be configured to continuously or substantially continuously monitor the vital signs of the patient 1802 and, upon determination that treatment is required, can be configured to deliver one or more therapeutic electrical pulses to the patient 1802. For example, such therapeutic shocks can be pacing, defibrillation, cardioversion, or transcutaneous electrical nerve stimulation (TENS) pulses.

The garment-based medical device 1800 can include one or more of the following: a garment 1810, one or more sensing electrodes 1812 (e.g., ECG electrodes), one or more therapy electrodes 1814a and 1814b (collectively referred to herein as therapy electrodes 1814), a medical device controller 1820, a connection pod 1830, a patient interface pod 1840, a belt 1850, or any combination of these. In some examples, at least some of the components of the garment-based medical device 1800 can be configured to be affixed to the garment 1810 (or in some examples, permanently integrated into the garment 1810), which can be worn about the patient's torso.

The medical device controller 1820 can be operatively coupled to the sensing electrodes 1812, which can be affixed to the garment 1810 (e.g., assembled into the garment 1810 or removably attached to the garment 1810, for example, using hook-and-loop fasteners). In some implementations, the sensing electrodes 1812 can be permanently integrated into the garment 1810. The medical device controller 1820 can be operatively coupled to the therapy electrodes 1814. For example, the therapy electrodes 1814 can also be assembled into the garment 1810, or, in some implementations, the therapy electrodes 1814 can be permanently integrated into the garment 1810.

Component configurations other than those shown in FIG. 18 are possible. For example, the sensing electrodes 1812 can be configured to be attached at various positions about the body of the patient 1802. The sensing electrodes 1812 can be operatively coupled to the medical device controller 1820 through the connection pod 1830. In some implementations, the sensing electrodes 1812 can be adhesively attached to the patient 1802. In some implementations, the sensing electrodes 1812 and at least one of the therapy electrodes 1814 can be included on a single integrated patch and adhesively applied to the patient's body.

The sensing electrodes 1812 can be configured to detect one or more cardiac signals. Examples of such signals include ECG signals and/or sensed cardiac physiological signals from the patient 1802. In certain implementations, the sensing electrodes 1812 can include additional components such as accelerometers, acoustic signal detecting devices, and other measuring devices for recording additional parameters. For example, the sensing electrodes 1812 can also be configured to detect other types of patient physiological parameters and acoustic signals, such as tissue fluid levels, heart vibrations, lung vibrations, respiration vibrations, patient movement, etc. Example sensing electrodes 1812 include a metal electrode with an oxide coating such as tantalum pentoxide electrodes, as described in, for example, U.S. Pat. No. 6,253,099 entitled “Cardiac Monitoring Electrode Apparatus and Method,” the content of which is incorporate herein by reference.

In some examples, the therapy electrodes 1814 can also be configured to include sensors configured to detect ECG signals as well as other physiological signals of the patient 1802. The connection pod 1830 can, in some examples, include a signal processor configured to amplify, filter, and digitize these cardiac signals prior to transmitting the cardiac signals to the medical device controller 1820. One or more of the therapy electrodes 1814 can be configured to deliver one or more therapeutic defibrillating shocks to the body of the patient 1802 when the garment-based medical device 1800 determines that such treatment is warranted based on the signals detected by the sensing electrodes 1812 and processed by the medical device controller 1820. Example therapy electrodes 1814 can include conductive metal electrodes such as stainless-steel electrodes that include, in certain implementations, one or more conductive gel deployment devices configured to deliver conductive gel to the metal electrode prior to delivery of a therapeutic shock.

In some implementations, a garment-based medical device as described herein can be configured to switch between a therapeutic medical device and a monitoring medical device that is configured to only monitor a patient (e.g., not provide or perform any therapeutic functions). For example, therapeutic components such as the therapy electrodes 1814 and associated circuitry can be decoupled from (or coupled to) or switched out of (or switched into) the garment-based medical device. As an illustration, a garment-based medical device can have optional therapeutic elements (e.g., defibrillation and/or pacing electrodes components, and associated circuitry) that are configured to operate in a therapeutic mode. The optional therapeutic elements can be physically decoupled from the garment-based medical device as a means to convert the therapeutic garment-based medical device into a monitoring garment-based medical device for a specific use (e.g., for operating in a monitoring-only mode) for a patient. Alternatively, the optional therapeutic elements can be deactivated (e.g., by means or a physical or a software switch), essentially rendering the therapeutic garment-based medical device as a monitoring garment-based medical device for a specific physiologic purpose or a particular patient. As an example of a software switch, an authorized person can access a protected user interface of the garment-based medical device and select a preconfigured option or perform some other user action via the user interface to deactivate the therapeutic elements of the garment-based medical device.

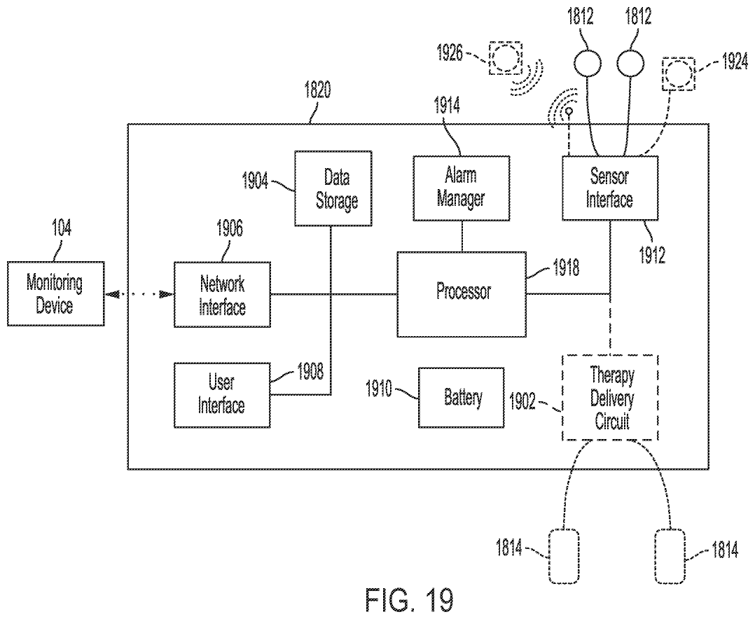

FIG. 19 illustrates a sample component-level view of the medical device controller 1820. As shown in FIG. 19, the medical device controller 1820 can include a therapy delivery circuit 1902, a data storage 1904, a network interface 1906, a user interface 1908, at least one battery 1910, a sensor interface 1912, an alarm manager 1914, and at least one processor 1918. As described above, in some implementations, the garment-based medical device 1800 may not deliver therapy and instead may be used only for monitoring the patient 1802. As such, a monitoring garment-based medical device 1800 can include a medical device controller 1820 that includes like components as those described above but does not include a therapy delivery circuit 1902 (shown in dotted lines).

The therapy delivery circuit 1902 can be coupled to the therapy electrodes 1814 configured to provide therapy to the patient 1802. For example, the therapy delivery circuit 1902 can include, or be operably circuitry components that are configured to generate and provide the therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuit and under control of one or more processors (e.g., processor 1918) to provide, for example, one or more pacing, defibrillation, or cardioversion therapeutic pulses.

Pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation or cardioversion pulses can be used to treat ventricular tachycardia and/or ventricular fibrillation.

The capacitors can include a parallel-connected capacitor bank consisting of a plurality of capacitors (e.g., two, three, four or more capacitors). These capacitors can be switched into a series connection during discharge for a defibrillation pulse. For example, four capacitors of approximately 650 μF can be used. The capacitors can have between 350 to 500 V surge rating and can be charged in approximately 15 to 30 seconds from a battery pack.

For example, each defibrillation pulse can deliver between 60 to 180 J of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 J) regardless of the patient's body impedance. The therapy delivery circuit 1902 can be configured to perform the switching and pulse delivery operations, e.g., under control of the processor 1918. As the energy is delivered to the patient 1802, the amount of energy being delivered can be tracked. For example, the amount of energy can be kept to a predetermined constant value even as the pulse waveform is dynamically controlled based on factors such as the patient's body impedance which the pulse is being delivered.

The data storage 1904 can include one or more of non-transitory computer readable media, such as flash memory, solid state memory, magnetic memory, optical memory, cache memory, combinations thereof, and others. The data storage 1904 can be configured to store executable instructions and data used for operation of the medical device controller 1820. In certain implementations, the data storage can include executable instructions that, when executed, are configured to cause the processor 1918 to perform one or more functions.

In some examples, the network interface 1906 can facilitate the communication of information between the medical device controller 1820 and one or more other devices or entities over a communications network. For example, the network interface 1906 can be configured to communicate with the remote server 102 or other similar computing device. The network interface 1906 can include communications circuitry for transmitting data in accordance with a Bluetooth® wireless standard for exchanging such data over short distances to an intermediary device(s) (e.g., the portable gateway 108 or another base station, “hotspot” device, smartphone, tablet, portable computing device, and/or other device in proximity of the garment-based medical device 1800). The intermediary device(s) may in turn communicate the data to the remote server 102 over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server 102 over a Wi-Fi communications link based on the IEEE 802.11 standard.

In certain implementations, the user interface 1908 can include one or more physical interface devices such as input devices, output devices, and combination input/output devices and a software stack configured to drive operation of the devices. These user interface elements may render visual, audio, and/or tactile content. Thus, the user interface 1908 may receive input or provide output, thereby enabling a user to interact with the medical device controller 1820.

The medical device controller 1820 can also include at least one battery 1910 configured to provide power to one or more components integrated in the medical device controller 1820. The battery 1910 can include a rechargeable multi-cell battery pack. In one example implementation, the battery 1910 can include three or more 2200 mA lithium ion cells that provide electrical power to the other device components within the medical device controller 1820. For example, the battery 1910 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 1820.

The sensor interface 1912 can be coupled to one or more sensors configured to monitor one or more physiological parameters of the patient. As shown, the sensors may be coupled to the medical device controller 1820 via a wired or wireless connection. The sensors can include one or more sensing electrodes 1812 (e.g., ECG electrodes). In some embodiments, as further shown in FIG. 19, the sensors may include additional sensors, such as heart vibrations sensors 1924 and tissue fluid monitors 1926 (e.g., based on ultra-wide band radiofrequency devices), which are not shown in FIG. 18. The sensor interface 1912 can be coupled to any one or combination of sensing electrodes/other sensors to receive other patient data indicative of patient parameters. Once data from the sensors has been received by the sensor interface 1912, the data can be directed by the processor 1918 to an appropriate component within the medical device controller 1820. For example, if heart data is collected by the heart vibrations sensor 1924 and transmitted to the sensor interface 1912, the sensor interface 1912 can transmit the data to the processor 1918 which, in turn, relays the data to a cardiac event detector. The cardiac event data can also be stored on the data storage 1904.

In certain implementations, the alarm manager 1914 can be configured to manage alarm profiles and notify one or more intended recipients of events, where an alarm profile includes a given event and the intended recipients who may have an interest in the given event. These intended recipients can include external entities, such as users (e.g., patients, physicians and other caregivers, a patient's loved one, monitoring personnel), as well as computer systems (e.g., monitoring systems or emergency response systems, which may be included in the remote server 102 or may be implemented as one or more separate systems). The alarm manager 1914 can be implemented using hardware or a combination of hardware and software. For instance, in some examples, the alarm manager 1914 can be implemented as a software component that is stored within the data storage 1904 and executed by the processor 1918. In this example, the instructions included in the alarm manager 1914 can cause the processor 1918 to configure alarm profiles and notify intended recipients using the alarm profiles. In other examples, the alarm manager 1914 can be an application-specific integrated circuit (ASIC) that is coupled to the processor 1918 and configured to manage alarm profiles and notify intended recipients using alarms specified within the alarm profiles. Thus, examples of the alarm manager 1914 are not limited to a particular hardware or software implementation.

In some implementations, the processor 1918 includes one or more processors (or one or more processor cores) that each are configured to perform a series of instructions that result in the manipulation of data and/or the control of the operation of the other components of the medical device controller 1820. In some implementations, when executing a specific process (e.g., cardiac monitoring), the processor 1918 can be configured to make specific logic-based determinations based on input data received. The processor 1918 may be further configured to provide one or more outputs that can be used to control or otherwise inform subsequent processing to be carried out by the processor 1918 and/or other processors or circuitry with which the processor 1918 is communicatively coupled. Thus, the processor 1918 reacts to a specific input stimulus in a specific way and generates a corresponding output based on that input stimulus. In some example cases, the processor 1918 can proceed through a sequence of logical transitions in which various internal register states and/or other bit cell states internal or external to the processor 1918 may be set to logic high or logic low. As referred to herein, the processor 1918 can be configured to execute a function where software is stored in a data store coupled to the processor 1918, the software being configured to cause the processor 1918 to proceed through a sequence of various logic decisions that result in the function being executed. The various components that are described herein as being executable by the processor 1918 can be implemented in various forms of specialized hardware, software, or a combination thereof. For example, the processor 1918 can be a digital signal processor (DSP) such as a 24-bit DSP processor. As another example, the processor 1918 can be a multi-core processor, e.g., having two or more processing cores. As another example, the processor can be an Advanced RISC Machine (ARM) processor, such as a 32-bit ARM processor. The processor 1918 can execute an embedded operating system and further execute services provided by the operating system, where these services can be used for file system manipulation, display and audio generation, basic networking, firewalling, data encryption, communications, and/or the like.

Referring back to FIG. 18, the monitoring device 104 is configured to be attached to the garment 1810. For example, as further illustrated in FIG. 18, the garment 1810 may include a strap 1860 configured to cross across the patient's chest. The monitoring device 104 may therefore be attached to the strap 1860 above the patient's sternum (e.g., similar to how the monitoring device 104 may be mounted on the band 1100, as described above with reference to FIG. 11). The strap 1860 may be made of an elastic and/or compressive material such that the strap 1860 fits close to the thorax of the patient 1802, allowing the monitoring device 104 to be mounted against or nearly against the patient's skin. The monitoring device 104 may include an RF transmitter and RF receiver, as described above. The monitoring device 104 may further include one or more light sources and a light sensor and/or the strap 1860 may include the one or more light sources and light sensor (e.g., similar to the monitoring device 104 and the band 1100 as described above with reference to FIG. 11).

As further shown in FIG. 19, the monitoring device 104 may be configured to communicate with the medical device controller 1820. For example, in some implementations, the medical device controller 1820 may provide instructions to the monitoring device 104 to control operation of the monitoring device 104. In some implementations, the monitoring device 104 may be controlled based on instructions stored at the monitoring device 104 and may instead exchange data with the medical device controller 1820 (e.g., transmit RF sensor signals and light sensor signals to the medical device controller 1820). To facilitate communication between the monitoring device 104 and the medical device controller 1820, in some implementations, the garment 1810 may include internal wiring that allows the monitoring device 104 to communicate with the medical device controller 1820 when the monitoring device 104 is mounted onto the garment-based medical device 1800 as shown in FIG. 18. In some implementations, the monitoring device 104 may include wiring (not shown) configured to connect to the medical device controller 1820. In some implementations, the monitoring device 104 may communicate wirelessly with the medical device controller 1820. For example, the monitoring device 104 may communicate directly (e.g., via Bluetooth®, Wi-Fi, radio-frequency identification (RFID), NFC, Body Area Network, etc.) with the medical device controller 1820, such as in embodiments of the cardiovascular monitoring unit 100 that do not include a portable gateway 108. As another example, the cardiovascular monitoring unit 100 may include a portable gateway 108, and the monitoring device 104 may communicate with the medical device controller 1820 via the portable gateway. In some implementations, the monitoring device 104 may not communicate with the medical device controller 1820 and may instead communicate with the remote server 102 (e.g., directly, via Wi-Fi or cellular networks, or indirectly via the portable gateway 108).

Alternatively, in some embodiments, a cardiovascular monitoring unit 100 may include a garment-based medical device (e.g., similar to the garment-based medical device 1800) but not a monitoring device 104. Instead, the functionalities of the monitoring device 104 described above may be integrated into the garment-based medical device. For instance, the garment-based medical device may include a strap (e.g., similar to the strap 1860), where an RF transmitter, RF receiver, at least one light source, and light sensor (along with associated circuitry and/or power source(s), as needed) are permanently or removably attached to the strap. The RF transmitter, RF receiver, at least one light source, and light sensor may have a wired or wireless connection to the medical device controller 1820, which controls the functionality of the RF transmitter, RF receiver, at least one light source, and light sensor.

The embodiments of a cardiovascular monitoring unit 100 shown in FIGS. 1-19 and described above are examples, and other embodiments of a cardiovascular monitoring unit 100 may be contemplated herein. As an illustration, in some implementations, a cardiovascular monitoring unit 100 may not include an adhesive patch, wearable combination piece, garment-based medical device, or other mechanism for holding a monitoring device (e.g., the monitoring device 104) against the patient's thorax. Instead, the monitoring device may be configured for temporary use. For example, the monitoring device may be configured to be held against a first location on the patient (e.g., the patient's thorax over their sternum) for a certain amount of time while the monitoring device transmits and receives RF waves and light waves to and from the patient's thorax. Once the monitoring device has produced sufficient RF sensor signals and light sensor signals (e.g., RF and light sensor signals of at least a certain threshold of amplitude and length and having below a certain threshold of artifacts), the monitoring device may notify the user that the monitoring device can be removed. For instance, the monitoring device may emit a beep and/or change a light to indicate that the monitoring device has produced sufficient RF sensor signals and light sensor signals. The monitoring device may then transmit the RF sensor signals and light sensor signals to the remote server 102 and/or analyze the RF sensor signals and light sensor signals. The monitoring device may also facilitate the user in placing the monitoring device on the first location on the patient (e.g., the patient's sternum) such that the sufficient signals may be produced. To illustrate, the monitoring device may provide verbal instructions, light up visual indicators, provide beeps, and/or the like to help the user with placing and holding the monitoring device over the patient's sternum. Additionally, features of the cardiovascular monitoring units 100 shown in FIGS. 1-19 may be altered, combined, or switched out, in some embodiments. As an illustration, each of the cardiovascular monitoring units 100 may include ECG electrodes similar to the ECG electrodes 114 shown and described with respect to FIGS. 1, 2, and 6.

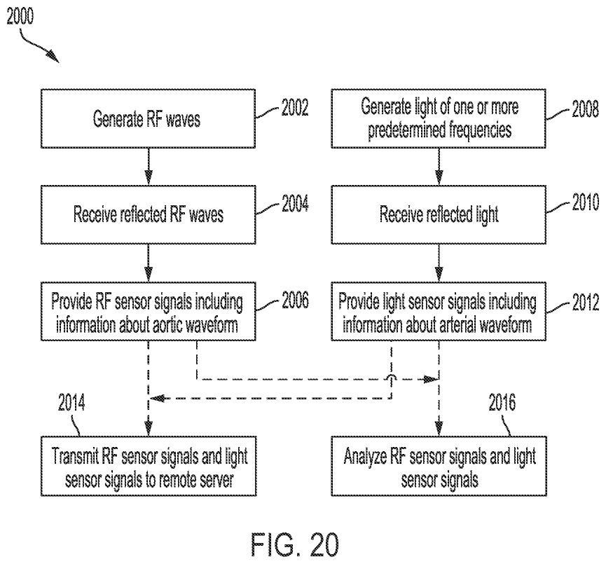

Referring now to FIG. 20, a sample process flow is shown whereby a cardiovascular monitoring unit provides RF sensor signals and light sensor signals. The sample process 2000 shown in FIG. 20 can be implemented by the RF transmitter, RF sensor, at least one light source, light sensor, and associated circuitry of a cardiovascular monitoring unit 100. For example, a monitoring device 104 may implement the sample process 2000 (e.g., via the microcontroller 606 of FIG. 6), as described in further detail below, though it should be understood that the sample process 2000 may be implemented via any of the embodiments of a cardiovascular monitoring unit 100 described herein or their equivalents.

As shown in FIG. 20, the monitoring device 104 generates RF waves at step 2002. For example, the monitoring device 104 may generate RF waves as described above with respect to FIGS. 6 and 7. In various implementations, as discussed above, the RF transmitter of the monitoring device 104, adhesive patch 106, garment-based medical device 1800, etc. is placed on the thorax of a patient (e.g., directly against or near, such as with another material in between that the RF waves can travel through) such that the generated RF waves are directed towards the patient's aortic region. The monitoring device 104 then receives RF waves reflected/scattered from the patient's aortic region at step 2004.

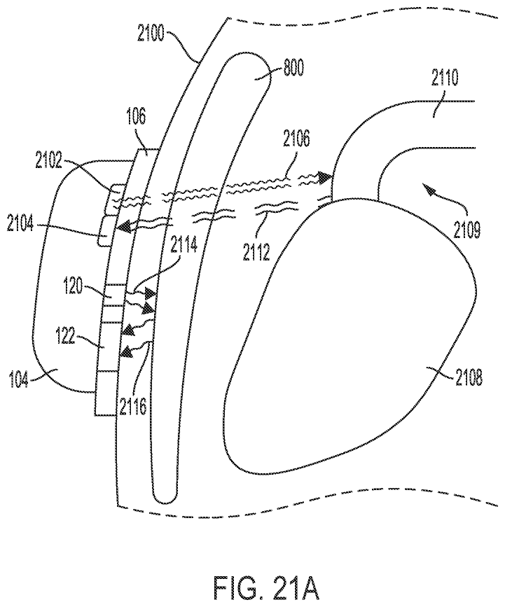

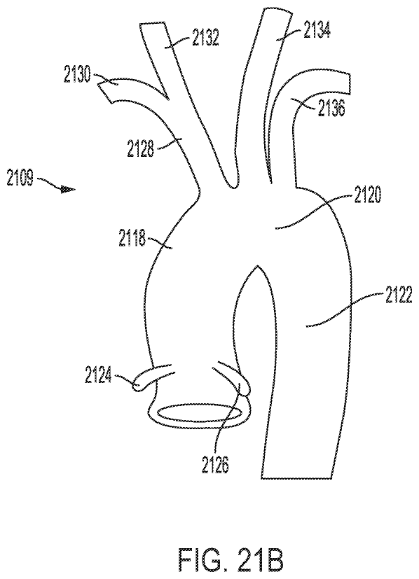

As an illustration, referring to FIG. 21A, an embodiment of a cardiovascular monitoring unit 100 being used on a patient is shown. For example, an adhesive patch 106 with an embedded light source 120 and light sensor 122 (e.g., similar to the embodiment of the adhesive patch 106 shown in FIG. 2) has been applied to a thorax 2100 of the patient above the patient's sternum 800. A monitoring device 104 (e.g., similar to the embodiment of the monitoring device 104 shown in FIG. 3) has been attached to the adhesive patch 106. The monitoring device 104 includes an RF transmitter 2102 and an RF receiver 2104 (e.g., which are shown as larger components in FIG. 21A but may, in some implementations, be flat components printed as part of a printed circuit board). As illustrated in FIG. 21A, the RF transmitter 2102 is configured to transmit RF waves 2106 through the patient's thorax 2100 in the general direction of the patient's heart 2108. In particular, the RF transmitter 2102 may transmit RF waves 2106 to an aortic region 2109 around the patient's aorta 2110, as shown in FIG. 20. At least some of the transmitted RF waves 2106 may be scattered or reflected by the arteries in patient's aortic region 2109, and reflected RF waves 2112 may be received at the RF receiver 2104.

In embodiments, the aortic region 2109 may include the patient's aorta 2110 and/or one or more arteries that branch off of the aorta 2110 and are proximate to the aorta 2110. To illustrate, FIG. 21B shows an example of the aortic region 2109. As shown in FIG. 21B, the aortic region 2109 may include the patient's ascending aorta 2118, aortic arch 2120, and/or descending aorta 2122. Alternatively or additionally, the aortic region 2109 may include one or more of the arteries branching off of the ascending aorta 2118, aortic arch 2120, and descending aorta 2122, such as the patient's right coronary artery 2124, left coronary artery 2126, brachiocephalic artery 2128, right subclavian artery 2130, right common carotid artery 2132, left common carotid artery 2134, and/or left subclavian artery 2136.

Referring back to FIG. 20, the monitoring device 104 provides RF sensor signals based on the received RF waves at step 2006. For example, the RF receiver and associated circuitry (e.g., as discussed above with respect to FIGS. 6-7) may generate RF sensor signals based on the received reflected RF waves. These generated RF sensor signals may contain information about an aortic region waveform of the patient, where the aortic region waveform correlates with the volume of the arteries in the patient's aortic region (e.g., the patient's aorta, the patient's brachiocephalic artery, and/or so on) over time.

Separately, as further shown in FIG. 20, the monitoring device 104 generates light of one or more predetermined frequencies at step 2008. For instance, the monitoring device 104 may include or be connected to at least one light source, such as at least one LED, where each light source is configured to generate light of a predetermined frequency. As an example, the monitoring device 104 may include or be connected to one or more red LEDs, one or more green LEDs, or one or more red and green LEDs. In various implementations, as discussed above, the at least one light source of the monitoring device 104, adhesive patch 106, garment-based medical device 1800, etc. is placed on the thorax of a patient (e.g., directly against or near, such as with another material in between that the generated light waves can travel through) such that the generated light is directed towards one or more arteries below skin on the thorax of the patient. To illustrate, the at least one light source may direct the light of the one or more predetermined frequencies to the surface arteries in or near the skin over the patient's thorax. The monitoring device 104 then receives light reflected/scattered by the patient's thorax at step 2010.

As an illustration, referring back to FIG. 21A, the light source 120 is configured to transmit light waves 2114 into the patient's thorax 2100. Specifically, as shown in FIG. 21A, the light source 120 may transmit the light waves 2114 into the patient's skin above the sternum 800, which contains one or more arteries. The light waves 2114 are reflected off of the patient's sternum 800, and these reflected light waves 2116 are received by the light sensor 122. The light sensor 122 and associated circuitry (e.g., discussed above with respect to FIG. 6) may generate light sensor signals based on the received reflected light waves 2116. These light sensor signals may contain information about an arterial waveform of the patient, where the arterial waveform correlates with the volume of the arteries near the skin surface over time.

Referring again to FIG. 20, the monitoring device 104 provides light sensor signals based on the received light waves at step 2012. For instance, the light sensor and associated circuitry (e.g., as discussed above with respect to FIG. 6) may generate light sensor signals based on the received reflected light waves. These generated light sensor signals may contain information about an arterial waveform of the patient, where the arterial waveform correlates with the volume of one or more of the patient's arteries below skin on the thorax of the patient.

As shown, in some implementations, the monitoring device 104 may transmit the RF sensor signals and light sensor signals to the remote server 102 at step 2014. The monitoring device 104 may transmit the RF sensor signals and light sensor signals to the remote server 102 via the portable gateway 108, in some implementations. In some implementations, the cardiovascular monitoring unit 100 may not include a portable gateway 108, and the monitoring device 104 may transmit the RF sensor signals and light sensor signals directly to the remote server 102.

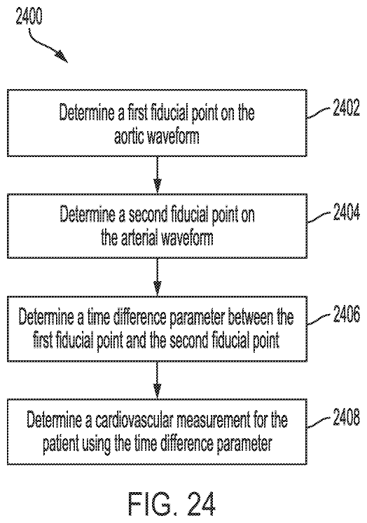

In some implementations, the monitoring device 104 may, additionally or alternatively, analyze the RF sensor signals and light sensor signals at step 2016. For example, the monitoring device 104 may accordingly carry out the process shown in FIG. 24, described in further detail below.

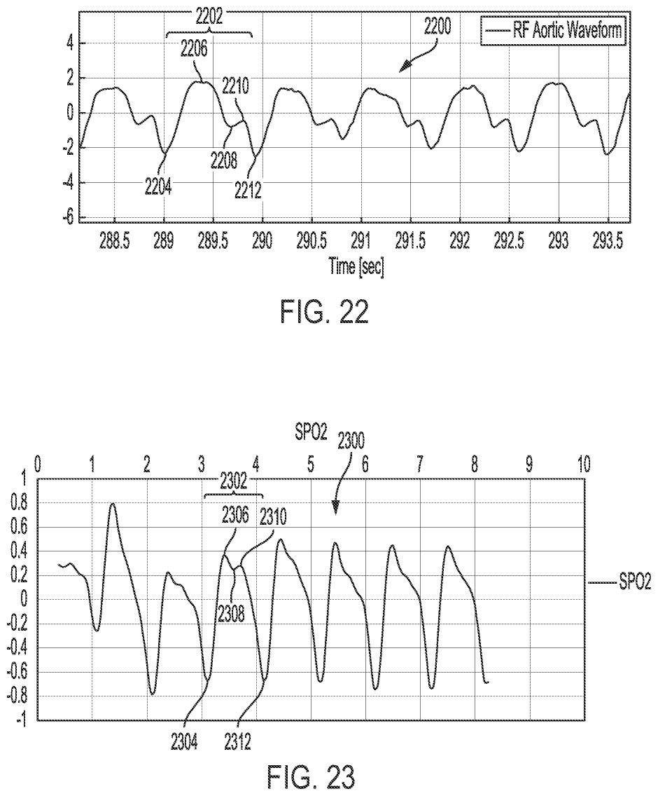

FIGS. 22 and 23 show example waveforms produced from the RF and light sensor signals (e.g., provided by the monitoring device 104 at steps 2006 and 2012 of FIG. 20). FIG. 22 illustrates an example aortic region waveform 2200 of the RF sensor signal amplitude over time (in seconds). The aortic region waveform 2200 may represent, for example, the volume of the patient's aorta and/or one or more arteries branching off from and proximate to the patient's aorta, such as the brachiocephalic artery. The aortic region waveform 2200 may include a number of fiducial points over a given cardiac cycle. For example, fiducial point 2204 occurs at the onset of the cardiac cycle 2202 (and the onset of the primary aortic region peak of the cardiac cycle 2202). Fiducial point 2204 also corresponds with the opening of the aortic valve and beginning of ventricular ejection and systole. Fiducial point 2206 occurs at the peak of the RF sensor signal over the cardiac cycle 2202 (e.g., the apex of the primary aortic region peak of the aortic region waveform 2200 over the cardiac cycle 2202) and corresponds with the peak systolic pressure in the arteries of the aortic region. Fiducial point 2208 occurs at the dicrotic notch of the cardiac cycle 2202 (e.g., at the end of the primary aortic region peak and the onset of the secondary aortic region peak), which corresponds with the closing of the aortic valve and the beginning of diastole. Fiducial point 2210 occurs at the apex of the secondary aortic region peak of the cardiac cycle 2202, and fiducial point 2212 occurs at the end of the cardiac cycle 2202 (and the end of the secondary aortic region peak) and the beginning of the next cardiac cycle.

FIG. 23 illustrates an example arterial waveform 2300 of the light signal amplitude over time (in seconds). Similar to the aortic region waveform 2200, the arterial waveform 2300 may include a number of fiducial points over a given cardiac cycle. As an example, fiducial point 2304 occurs at the onset of the cardiac cycle 2302 (and the onset of the primary arterial peak of the cardiac cycle 2302). Fiducial point 2304 also corresponds with the arrival of the arterial pulse wave, caused by the contraction of the left ventricle, at the surface arteries. Fiducial point 2306 occurs at the peak of the light sensor signal over the cardiac cycle 2302 (e.g., the apex of the primary arterial peak of the arterial waveform over the cardiac cycle 2302) and corresponds with the peak systolic pressure in the surface arteries. Fiducial point 2308 occurs at the dicrotic notch of the cardiac cycle 2302 (e.g., at the end of the primary arterial peak and the onset of the secondary arterial peak), which corresponds with the beginning of diastole in the surface arteries. Fiducial point 2312 occurs at the apex of the secondary arterial peak of the cardiac cycle 2302, and fiducial point 2312 occurs at the end of the cardiac cycle 2302 (and the end of the secondary arterial peak) and the beginning of the next cardiac cycle.

However, these fiducial points discussed with respect to FIGS. 22 and 23 are intended to be examples; other fiducial points may be identified on the aortic region waveform 2200 and the arterial waveform 2300. For instance, a fiducial point may be a local maximum or a local minimum of the aortic region waveform 2200 or the arterial waveform 2300. As another example, a fiducial point may be a point on the slope of the aortic region waveform or the arterial waveform 2300 (e.g., the halfway point on the slope as determined by the amplitude of the RF sensor signal or light sensor signal or as determined by the time of the slope, an inflection point of the slope, and so on).

In some embodiments, the monitoring device 104 (or equivalent discussed herein) may gate when RF and/or light measurements are taken, for example, to save battery power. For example, the monitoring device 104 may use ECG signals (e.g., from the ECG electrodes 114) to determine when to take RF measurements, such as by taking RF measurements only when the monitoring device 104 determines that the ECG signals are clean (e.g., having a signal amplitude of a certain level and free or relatively free of artifacts). As another example, the monitoring device 104 may use accelerometer signals to detect when the patient is active and use periods of activity to determine when to take RF and/or light measurements. Accordingly, the monitoring device 104 may then take RF and/or light measurements when the patient is inactive, and/or filter out RF and/or light measurements taken while the patient was active and the measurements are less likely to be clear. As another illustration, the monitoring device 104 may use the accelerometer signals (and in some implementations, additional signals such as ECG signals) to determine when the patient is sleeping. The monitoring device 104 may then take RF and/or light measurements when the patient is determined to be asleep. In some embodiments, instead of the monitoring device 104 gating measurements and/or filtering out measurements that are less likely to be clear, the remote server 102 may use ECG signals, accelerometer signals, and/or the like to identify the best quality RF sensor signals and light sensor signals. For instance, the remote server 102 may use accelerometer signals recorded by the monitoring device 104 to determine when the patient was asleep and perform an analysis (as described in further detail below) on the RF sensor signals and light sensor signals recorded during this period when the patient was asleep.

In some embodiments, the monitoring device 104 (or equivalent discussed herein) may take additional measurements that may affect the interpretation of the patient's cardiovascular measurements. As an example, the monitoring device 104 may use accelerometer or other posture sensor signals to detect the orientation or posture of a patient and transmit the posture sensor signals with the RF and light sensor signals to the remote server 102. As another example, the monitoring device 104 may use accelerometer or other respiration sensor signals to detect the respiration rate of the patient and transmit the respiration sensor signals to the remote server 102. The remote server 102 may use the additional measurements or signals, for instance, in preparing reports on the patient's cardiovascular health.

In some implementations, the monitoring device 104 may take RF measurements and/or light measurements depending on information about the patient determined from one or more of the additional signals. For example, the monitoring device 104 may determine using accelerometer signals (or, in some cases, the remote server 102 may determine using the accelerometer signals) that the patient is active. The monitoring device 104 may, for instance, determine that the patient is active based on accelerometer counts recorded in the accelerometer signals being above a certain threshold. The monitoring device 104 or remote server 102 may accordingly identify that an activity episode has ended based on the accelerometer signals, and the monitoring device 104 may take RF measurements and/or light measurements during this rest period immediately following the activity episode. Taking measurements during the rest period may allow the monitoring device 104 to have a higher likelihood of recording measurements correlated with blood pressure changes, which may be valuable information for caregivers. As another example, the monitoring device 104 (or, in some cases, the remote server 102) may determine from accelerometer signals that the patient is asleep. The monitoring device 104 may thus record RF measurements and/or light measurements while the patient is asleep, as these measurements may be more reflective of the patient's resting blood pressure than while the patient is awake.

Referring now to FIG. 24, a sample process flow is shown whereby a cardiovascular monitoring unit and/or a remote server determines a cardiovascular measurement for a patient. To illustrate, the sample process 2400 shown in FIG. 24 can be implemented by the cardiovascular monitoring unit 100 and/or by the remote server 102. For example, a monitoring device 104 may implement the sample process 2400 (e.g., via the microcontroller 606 of FIG. 6), as described in further detail below, though it should be understood that the sample process 2400 may be implemented via any of the embodiments of a cardiovascular monitoring unit 100 described herein or their equivalents. Moreover, the remote server 102 may alternatively or additionally implement the sample process 2400.

As shown in FIG. 24, the monitoring device 104 and/or the remote server 102 determines a first fiducial point on the aortic region waveform at step 2402. For example, the monitoring device 104 and/or remote server 102 may identify one of the fiducial points 2204-2212 of FIG. 22 as the fiducial point for the aortic region waveform. The monitoring device 104 and/or remote server 102 also determines a second fiducial point on the arterial waveform at step 2404. For example, the monitoring device 104 and/or remote server 102 may identify one of the fiducial points 2304-2312 of FIG. 23 as the fiducial point for the arterial waveform.

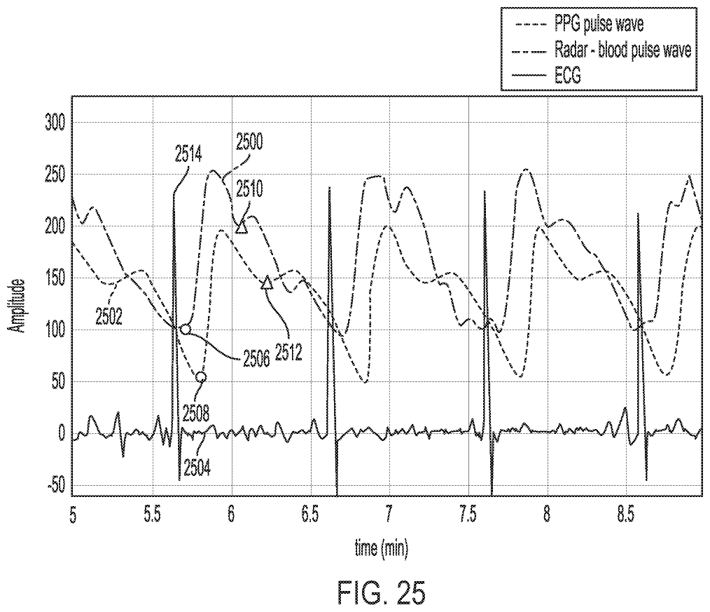

Once the monitoring device 104 and/or remote server 102 has identified the first and second fiducial points, the monitoring device 104 and/or remote server 102 determines a time difference parameter between the first and second fiducial points at step 2406. As an illustration, FIG. 25 shows an aortic region waveform 2500, plotted from an RF sensor signal, on the same timeline as an arterial waveform 2502, plotted from a light sensor signal, and an ECG waveform 2504, plotted from an ECG sensor signal (e.g., based on electrical activity of the heart sensed by electrodes 114). FIG. 25 also illustrates example fiducial points on the aortic region waveform 2500 and arterial waveform 2502. As shown in FIG. 25, the aortic region waveform 2500 and the arterial waveform 2502 have similar shapes, but the arterial waveform 2502 is delayed in time compared to the aortic region waveform 2500. This delay between the aortic region waveform 2500 and arterial waveform 2502 is because a pulse wave, caused by the aortic valve opening and ventricular ejection, will take some small amount of time to travel from the patient's aortic region near the heart to the arteries over the patient's thorax. Accordingly, there is a time difference between a fiducial point on the aortic region waveform 2500 and a corresponding fiducial point on the arterial waveform 2502.

As an example, the monitoring device 104 and/or remote server 102 may identify the first fiducial point on the aortic region waveform 2500 as fiducial point 2506, which occurs at the beginning of a cardiac cycle of the aortic region waveform 2500 and at the onset of the primary aortic region peak. The monitoring device 104 and/or remote server 102 may also identify the second fiducial point on the arterial waveform 2502 as fiducial point 2508, which similarly occurs at the beginning of a corresponding cardiac cycle of the arterial waveform 2502 and at the onset of the primary arterial peak. As another example, the monitoring device 104 and/or remote server 102 may identify the first fiducial point on the aortic region waveform 2500 as fiducial point 2510, which occurs at the dicrotic notch of the aortic region waveform 2512. The monitoring device 104 and/or remote server 102 may additionally identify the second fiducial point on the arterial waveform 2502 as fiducial point 2512, which also occurs at the dicrotic notch of the arterial waveform 2502. The monitoring device 104 and/or remote server 102 then determines the time difference parameter to be the difference between the times of the two fiducial points. Thus, referring to the previous examples, the monitoring device 104 and/or remote server may determine the time difference parameter as the time difference between fiducial points 2506 and 2508 or between fiducial points 2510 and 2512. In implementations, the time difference parameter may represent the pulse transit time (PTT) of the pulse wave moving from the aortic region to the surface arteries.

Returning to FIG. 24, after determining the time difference parameter, the monitoring device 104 and/or remote server 102 determines a cardiovascular measurement for the patient using the time difference parameter at step 2408. As an illustration, the monitoring device 104 and/or remote server 102 may determine that the beginning of a cardiac cycle on the aortic region waveform is the first fiducial point (e.g., fiducial point 2204 or fiducial point 2506) and the beginning of a corresponding cardiac cycle on the arterial waveform is the second fiducial point (e.g., fiducial point 2304 or fiducial point 2508). Therefore, the monitoring device 104 and/or remote server 102 may determine that the time difference parameter is the difference between the first and second fiducial points, with the time difference parameter also representing the PTT. This PTT may be the cardiovascular measurement for the patient. The PTT may range, for example, between 50 ms and 300 ms (e.g., depending on the age and health of the patient).

In some instances, the monitoring device 104 and/or remote server 102 may calculate the cardiovascular measurement using the time difference parameter. For instance, referring to the previous example, the monitoring device 104 and/or remote server 102 may divide the distance between the aortic region and the surface arteries along the arterial tree by the time difference parameter to find the pulse wave velocity (PWV), or the velocity of the pulse wave transmitted from the aortic region to the surface arteries. The PWV may range, for example, between 4 m/s and 22 m/s (e.g., depending on the age and health of the patient).

In some cases, the exact distance between the aortic region and the surface arteries along the arterial tree may be difficult to measure. As such, the distance between the aortic region and the surface arteries along the arterial tree may be approximated, in various implementations. The monitoring device 104 and/or remote server 102 may determine or receive the approximate distance between the aortic region and the surface arteries along the arterial tree through a number of different ways. In some implementations, the monitoring device 104 and/or remote server 102 may receive the approximate distance between the aortic region and the one or more arteries below the skin of the thorax from a caregiver. For example, caregiver input may be provided directly by a caregiver or other authorizer person, e.g., a technician or patient service representative, providing such input on behalf of the caregiver. To illustrate, the caregiver or patient service representative may manually measure the circumference of the patient's thorax and/or the patient's anteroposterior (AP) diameter. The caregiver or patient service representative may then enter the thorax circumference and/or anteroposterior diameter information directly into the monitoring device 104 via a user interface of the monitoring device 104, or via a separate device such as the portable gateway 108 (which, as previously noted, may be a cellular phone or a smartphone). As another example, the caregiver may enter the thorax circumference or anteroposterior diameter information to the remote server 102 (e.g., via a caregiver interface 118), and the remote server 102 may, in some cases, transmit the circumference and/or anteroposterior diameter to the monitoring device 104. In an example, the monitoring device 104 and/or remote server 102 halves the measured circumference to determine an approximation of the distance between the patient's aortic region and surface arteries along the arterial tree. Alternatively, the monitoring device 104 and/or remote server 102 may determine an approximation of the patient's thorax circumference from the anteroposterior diameter, such as by using the formula below, which assumes that the patient has typical upper torso physiology:

$$Chest wall circumference = 2 π \sqrt{\frac{{( AP diameter )}^{2} + {( \frac{7}{5} * AP diameter )}^{2}}{2}}$$

The monitoring device 104 and/or remote server 102 may then halve the determined circumference to approximate the distance between the aortic region and the surface arteries along the arterial tree.

In some implementations, the monitoring device 104 and/or remote server 102 may receive a body mass index (BMI) of the patient from the patient's caregiver (e.g., provided via the portable gateway 108 or a caregiver interface 118). The monitoring device 104 and/or remote server 102 may then use the patient's BMI to determine the approximate distance between the aortic region and the one or more arteries below the skin of the thorax along the arterial tree (e.g., by using a formula for the distance with BMI as an input, by using a table that gives the distance given the BMI and the patient's sex, etc.).

In some implementations, the monitoring device 104 may be further configured to transmit RF waves towards the patient's posterior thorax (e.g., to the spinal cord or other organs of the patient's posterior thorax) and receive reflected/scattered RF waves from the patient's posterior thorax. The monitoring device 104 may further provide second RF sensor signals based on the received RF waves reflected from the patient's posterior thorax. The monitoring device 104 and/or the remote server 102 may then use the second RF sensor signals to determine an anteroposterior diameter of the patient. The monitoring device 104 and/or remote server 102 can use the anteroposterior diameter to find the chest wall circumference (e.g., using the formula provided above) and halve the chest wall circumference to determine the approximate distance between the aortic region and the one or more arteries below the skin on the patient's thorax along the arterial tree.

In some implementations, the monitoring device 104 and/or the remote server 102 may also determine multiple cardiovascular measurements for the patient and further determine a summary cardiovascular measurement from the multiple cardiovascular measurements. For example, the monitoring device 104 and/or remote server 102 may determine the PTT or the PWV for each cardiac cycle within a summary time period by repeating the process 2400 described above for each cardiac cycle (e.g., identifying a number of first fiducial points on the aortic region waveform, identifying a number of second fiducial points on the arterial waveform, and determining a time difference parameter between each corresponding set of fiducial points). The summary time period may be measured in time (e.g., 5-10 s, 10-20 s, 20-30 s, 30-60 s, 60-90 s, 90-120 s, and/or the like), the summary time period may be measured as a number of cardiac cycles (e.g., 3-5 cardiac cycles, 5-10 cardiac cycles, 10-15 cardiac cycles, 15-20 cardiac cycles, and/or the like), the summary time period may be measured in a time that likely includes a certain number of cardiac cycles, and/or the like.

The monitoring device 104 and/or remote server 102 may then determine, for instance, a summary time difference parameter from the time difference parameters calculated for each of the cardiac cycles. For example, using the time difference parameters (e.g., PTTs) calculated for each of the cardiac cycles, the monitoring device 104 and/or remote server 102 may determine a mean time difference parameter, a median time difference parameter, a mode time difference parameter, a maximum time difference parameter, or another statistical measure. As another illustration, the monitoring device 104 and/or remote server 102 may determine a summary PWV from a PWV calculated for each of the cardiac cycles. For example, using the PWVs calculated for each of the cardiac cycles, the monitoring device 104 and/or remote server 102 may determine a mean PWV, a maximum PWV, a mode PWV, a minimum PWV, a maximum PWV, or another statistical measure.

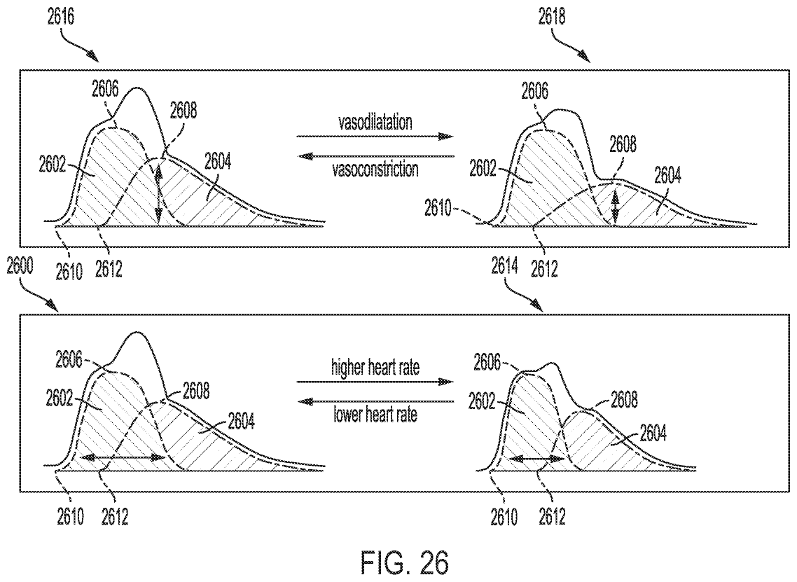

In some implementations, the monitoring device 104 and/or remote server 102 may determine a cardiovascular measurement differently from the process described with respect to FIG. 24. For instance, FIG. 26 illustrates additional aortic region or arterial waveforms. As shown, an aortic region or arterial waveform, such as waveform 2600, may be produced from the combination of an outgoing wave 2602, generated by the ventricular ejection, and a reflected wave 2604, reflected from bifurcations in the artery. As such, in some implementations, a monitoring device 104 and/or remote server 102 may parse out the outgoing wave 2602 and the reflected wave 2604 from the waveform 2600 (e.g., based on the shape of the waveform 2600). The monitoring device 104 and/or remote server 102 may then determine a time difference parameter using the outgoing wave 2602 and the reflected wave 2604.

As an example, the monitoring device 104 and/or remote server 102 may determine the difference in time between the apex 2606 of the outgoing wave 2602 and the apex 2608 of the reflected wave 2604 or between the onset 2610 of the outgoing wave 2602 and the onset 2612 of the reflected wave 2604. The difference in time between the outgoing wave 2602 and the reflected wave 2604 may be a cardiovascular measurement that may contain information, for example, about the blood pressure or heart rate of the patient. To illustrate, as shown by waveform 2614, the time difference between the apexes 2606 and 2608 and the onsets 2610 and 2612 may be affected by the patient's heart rate. Additionally, this time difference may be smaller for unhealthy patients with less elastic arterial walls, as the decreased elasticity may create a faster reflection from the artery bifurcations. As another example, the monitoring device 104 and/or remote server 102 may determine the difference in amplitude between the apex 2606 of the outgoing wave 2602 and the apex 2608 of the reflected wave 2604. The difference in height between the outgoing wave 2602 and the reflected wave 2604 may be a cardiovascular measurement that may contain information, for example, about the blood pressure of the patient or dilation of the patient's arteries. As an illustration, as shown by waveforms 2616 and 2618, the difference in height between the apexes 2606 and 2608 may be affected by whether the patient's arteries are vasodilated or vasoconstricted.

As another example, the monitoring device 104 and/or remote server 102 may identify the R-wave of a QRS complex in an ECG waveform (e.g., R-wave 2514 of ECG waveform 2504 of FIG. 25) and the onset of the pulse wave of the aortic region waveform (e.g., fiducial point 2506 of the aortic region waveform 2500). The ECG waveform may be produced from ECG signals provided by the monitoring device 104 (e.g., based on electrical activity of the heart sensed by ECG electrodes 114), and the aortic region waveform may be produced from RF sensor signals provided by the monitoring device 104. The monitoring device 104 and/or remote server 102 may then determine the time difference between the peak of the R-wave of the ECG waveform and the onset of the pulse wave of the RF-based aortic region waveform. This time difference parameter may represent, for example, the pre-ejection period (PEP) of the time between the electrical depolarization of the left ventricle and the beginning of ventricular ejection. In implementations, the monitoring device 104 and/or remote server 102 may add the PEP to the PTT to determine a pulse arrival time (PAT). Alternatively, or additionally, the monitoring device 104 and/or remote server 102 may determine the PAT as the difference in time between the peak of the R-wave and the onset of the pulse wave of the light-based arterial waveform.

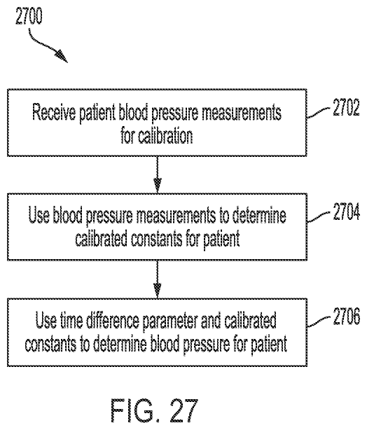

In some implementations, the time difference parameter and/or the cardiovascular measurement of FIG. 24 may be used to determine an additional cardiovascular measurement, such as the patient's blood pressure. As an illustration, FIG. 27 illustrates a sample process flow whereby a cardiovascular measurement and/or a remote server determines the patient's blood pressure. To illustrate, the sample process 2700 shown in FIG. 27 can be implemented by the cardiovascular monitoring unit 100 and/or by the remote server 102. For example, a monitoring device 104 may implement the sample process 2700 (e.g., via the microcontroller 606 of FIG. 6), as described in further detail below, though it should also be understood that the sample process 2700 may be implemented via any of the embodiments of a cardiovascular monitoring unit 100 described herein or their equivalents. Moreover, the remote server 102 may alternatively or additionally implement the sample process 2700.

As shown in FIG. 27, the monitoring device 104 and/or the remote server 102 receives patient blood pressure measurements for calibration at step 2702. For example, the patient's caregiver or a patient service representative may take a certain number of blood pressure measurements from the patient using a sphygmomanometer. These measurements may be taken at different time intervals (e.g., 30 seconds apart, one minute apart, two minutes apart, five minutes apart, etc.), at different heart rates (e.g., at the patient's resting heart rate, at light exercise, at medium exercise, and at heavy exercise), at different vasoconstriction levels (e.g., at no vasoconstriction, at light vasoconstriction, and at light vasodilation), and/or the like. The caregiver or patient service representative may then provide the blood pressure measurements to the monitoring device 104 and/or the remote server 102, such as via the portable gateway 108 or via a caregiver interface 118.

Once the monitoring device 104 and/or the remote server 102 receives the patient blood pressure measurements, at step 2704, the monitoring device 104 and/or the remote server 102 determines pre-calibrated constants for the patient that can be used to later determine the patient's blood pressure. As an illustration, equations for the patient's systolic blood pressure (Pₛ) and diastolic blood pressure (P_{d}) may be provided as follows:

Pₛ=A*ln(PTT)+B

P_{d}=C*ln(PTT)+D

In these equations, A and B are constants used to find the patient's systolic blood pressure, and C and D are constants used to find the patient's diastolic blood pressure. Accordingly, the monitoring device 104 and/or the remote server 102 may determine A, B, C, and D for the patient using systolic and diastolic blood pressure measurements received at step 2702, as well as PTT measurements that correspond to the received systolic and diastolic blood pressure measurements (e.g., taken by the monitoring device 104). For example, in some embodiments, the monitoring device 104 and/or remote server 102 may use curve fitting to determine A, B, C, and D using the systolic and diastolic blood pressure measurements.

The monitoring device 104 and/or remote server 102 then uses a later-determined time difference parameter (e.g., determined using the sample process 2400 of FIG. 24) and the calibrated constants to determine a blood pressure for the patient at step 2706. For example, the monitoring device 104 and/or remote server 102 may input the PTT into the above equations for systolic and diastolic blood pressure, using the calibrated constants from step 2704, to determine the systolic and diastolic blood pressure for the patient. In some implementations, the monitoring device 104 and/or remote server 102 may determine a summary PTT (e.g., a mean PTT from a certain number of cardiac cycles) and input the summary PTT into the above equations to determine the systolic and diastolic blood pressure. In some implementations, the monitoring device 104 and/or remote server 102 may determine a summary blood pressure for a summary time period, similar to the process of determining a summary time difference parameter and/or summary PWV discussed above.

Alternatively, in some implementations, the monitoring device 104 and/or remote server 102 may use a different process from the example process described above to determine the patient's blood pressure. For example, instead of being a natural logarithmic function as shown above, the function may be another type of logarithmic function, a linear function, a second-order polynomial function, a third-order polynomial function, a fourth-order polynomial function, an nth-order polynomial function, an exponential function, a quadratic function, and/or another type of predetermined function. The monitoring device 104 and/or the remote server 102 may determine constants for a selected function (e.g., similar to the process of determining A, B, C, and D described above). Alternatively, or additionally, the monitoring device 104 and/or the remote server 102 may determine a type of function that best fits the patient's blood pressure measurements received at step 2702 and PTT using a curve fitting process.

As another example, the monitoring device 104 and/or remote server 102 may use a function where the input is a different parameter from PTT. To illustrate, a patient's PWV may alternatively be represented by the Moens-Korteweg equation:

$$PWV = \sqrt{\frac{{hE}_{inc}}{2 ρ R}}$$

In the Moens-Korteweg equation, h is the artery wall thickness, E_{inc} is the arterial stiffness (e.g., Young's modulus), ρ is the blood density, and R is the artery radius. The Moens-Korteweg equation may be modified to provide the equation below that includes the patient's blood pressure (P):

$$PWV = \sqrt{\frac{{hE}_{0} {e}^{α ( P - {P}_{0} )}}{2 ρ R}}$$

In the above equation, E₀ is the arterial elasticity, and P₀ is a constant to calibrate the blood pressure. As such, the monitoring device 104 and/or remote server 102 may determine the constants above for the patient from the blood pressure measurements received at step 2702. Alternatively, or additionally, the monitoring device 104 and/or remote server 102 may determine the constants above for the patient based on alternative or additional measurements, such as PTT, PWV, tables for constants given the patient's physiological and/or biometric information (e.g., the patient's age, blood pressure, and pulse rate), and/or the like. The monitoring device 104 and/or remote server 102 may thus determine the patient's blood pressure as proportional to the natural logarithm of the square of the PWV.

The monitoring device 104 and/or remote server 102 may additionally, in some implementations, determine further cardiovascular measurements for the patient from the blood pressure. As an example, the monitoring device 104 and/or remote server 102 may determine the mean arterial pressure (MAP) for the patient using the following equation:

$$MAP = \frac{{P}_{s} + 2 {P}_{d}}{3}$$

Mean arterial pressure may be useful to a caregiver as a better indicator of perfusion to vital organs over systolic or diastolic pressure alone. As another example, the monitoring device 104 and/or remote server 102 may determine the patient's pulse pressure by subtracting the diastolic blood pressure from the systolic blood pressure. Knowing pulse pressure may help a caregiver determine when the patient is at risk for a negative heart event, such as a heart attack or stroke, as a higher pulse pressure (e.g., above 60 mmHg) may be correlated with stiff artery walls.

In some implementations, if the monitoring device 104 determines one or more cardiovascular measurements for a patient, the monitoring device 104 transmits the cardiovascular measurements to the remote server 102 (e.g., via the portable gateway 108). The remote server 102 may then prepare a report for a caregiver of the patient using the received cardiovascular measurements. Alternatively, or additionally, the remote server 102 may prepare a report using one or more cardiovascular measurements determined by the remote server 102. The report may further be prepared, in some cases, using input from a technician interface 116. For instance, a technician may indicate a time period to use for the report, the types of cardiovascular measurements to include in the report (e.g., blood pressure, PTT, PWV, etc.), whether to include individual measurements or summary measurements, a format for the report, and/or so on. Once the report is prepared, the remote server 102 transmits the report to a caregiver interface 118. In some cases, the caregiver may also be able to interact with the report via the caregiver interface 118, for example, to see additional data about waveforms or individual cardiovascular measurements associated with the report.

In some implementations, the monitoring device 104 and/or remote server 102 may monitor the patient's cardiovascular measurements over time. As an example, the monitoring device 104 and/or remote server 102 may determine whether the patient's cardiovascular measurements, such as the patient's blood pressure, PTT, and PWV, increase above or decrease below predetermined thresholds. These predetermined thresholds may be set according to the patient's age, gender, health, and so on. For example, for an eighty-year-old patient, the monitoring device 104 and/or remote server 102 may monitor the patient to determine if the patient's PWV increases above about 13 m/s (e.g., a 75th percentile PWV value for an eighty-year-old) and/or decreases below above 8.5 m/s (e.g., a 25th percentile PWV value for an eighty-year-old).

As another example, the monitoring device 104 and/or remote server 102 may set a baseline cardiovascular measurement for the patient. To illustrate, when the patient is provided with the cardiovascular monitoring unit 100, a technician or patient service representative may perform a baselining process for the patient. The baselining process may include, for example, taking an ECG from the patient, taking blood pressure measurements from the patient, measuring the patient's anteroposterior diameter, and so on. As such, for instance, a technician may be or assist a caregiver providing blood pressure measurements and a torso circumference or anteroposterior diameter to the monitoring device 104 and/or remote server 102 that the monitoring device 104 and/or remote server 102 can use to determine the patient's blood pressure, as described in further detail above. Using these measurements, the monitoring device 104 and/or remote server 102 may further set baseline cardiovascular measurements, such as a baseline blood pressure, PTT, PWV, and so on, that the monitoring device 104 and/or remote server 102 uses to monitor changes in the patient over time. Accordingly, the monitoring device 104 and/or remote server 102 may monitor the patient's cardiovascular measurements to determine if there is a predetermined percentage change from the baseline (e.g., a percentage deviation above or below the baseline), such as a 10% change, 15% change, 20% change, 25% change, 30% change, and so on.

In some implementations, if the monitoring device 104 and/or remote server 102 determines that the patient's cardiovascular measurements have increased above or below a predetermined threshold and/or show a predetermined percentage change from a baseline, the monitoring device 104 and/or remote server 102 may alert a caregiver for the patient. For example, the monitoring device 104 and/or remote server 102 may transmit an alert to a caregiver interface 118 associated with the patient's caregiver, with the alert indicating the increase above/decrease below the predetermined threshold and/or percentage change from the baseline. As another example, a technician interface 116 may receive the cardiovascular measurements showing the increase above/decrease below the predetermined threshold and/or predetermined percentage change from the baseline. As such, the technician interface 116 in communication with the remote server 102 may prepare a report for the patient's caregiver alerting the caregiver of the increase/decrease and/or percentage change, which the technician interface 116 or remote server 102 transmits to the caregiver interface 118.

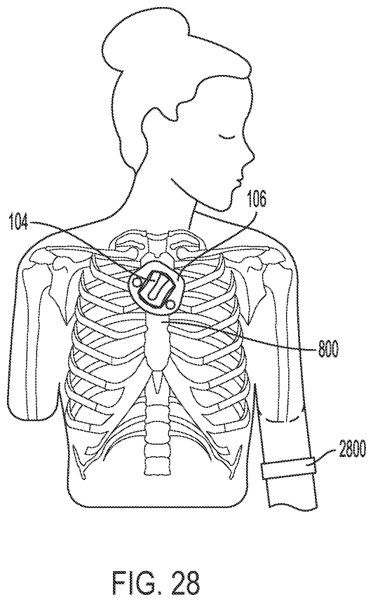

In some implementations, the monitoring device 104 and/or remote server 102 may determine one or more additional cardiovascular measurements for a patient using a secondary device positioned on the patient's body (e.g., at a second location from the monitoring device 104). The secondary device may provide, for example, a second set of RF signals including information about an artery at the second location of the patient, such as the patient's radial artery, brachial artery, or subclavian artery. As an illustration, FIG. 28 shows a patient using a cardiovascular monitoring unit 100 with a monitoring device 104 mounted on an adhesive patch 106 placed on the patient at a first location (e.g., over the patient's sternum) and an armband device 2800 placed on the patient at a second location (e.g., on the patient's wrist over the radial artery). For example, the armband device 2800 may include an elastic band, a strap with a hook-and-loop fastener on the ends, a strap with a snap on the ends, or so on to provide a close fit against the patient's wrist. The armband device 2800 also includes an RF transmitter and an RF receiver (e.g., similar to the RF transmitter and RF receiver incorporated into the monitoring device 104 and/or the adhesive patch 106, as discussed above). The armband device 2800 may thus provide a second set of RF sensor signals based on RF waves transmitted into and reflected from the patient's radial artery, where the second set of RF signals includes information about an RF-based radial waveform of the patient.

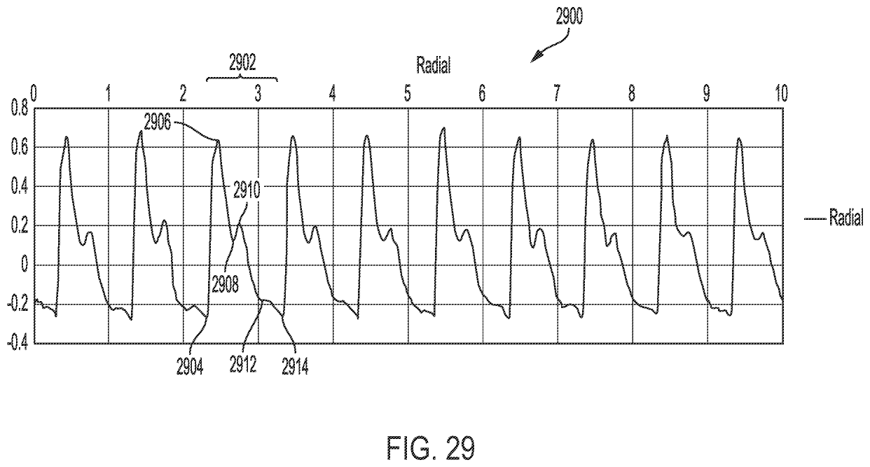

The radial waveform may be similar to the aortic region waveform (e.g., aortic region waveform 2200), having a peak associated with the radial artery opening in response to ventricular ejection. For instance, FIG. 29 illustrates an example radial waveform 2900 of the RF sensor signal from the armband device 2800 over time (in seconds). Similar to the aortic region waveform 2200, the radial waveform 2900 may include a number of fiducial points over a given cardiac cycle. For example, fiducial point 2904 occurs at the onset of the cardiac cycle 2902 (and the onset of the primary radial peak of the cardiac cycle 2902). Fiducial point 2906 occurs at the peak of the RF sensor signal over the cardiac cycle 2902. Fiducial point 2908 occurs at the dicrotic notch of the cardiac cycle 2902. Fiducial point 2910 occurs at the apex of the secondary radial peak of the cardiac cycle 2902. Fiducial point 2912 occurs where the slope of the secondary radial peak changes, and fiducial point 2914 occurs at the end of the cardiac cycle 2902 (and the end of the secondary radial peak) and the beginning of the next cardiac cycle. However, while the radial waveform 2900 has a similar shape to the aortic region waveform, the radial waveform 2900 is offset in time compared to the aortic region waveform because of the time that it will take a pulse wave to travel from the aortic region to the radial artery at the wrist.

The monitoring device 104 and/or remote server 102 may be in communication with the armband device 2800. As an example, the armband device 2800 may communication directly with the monitoring device 104 (e.g., using Bluetooth®, Wi-Fi, RFID, NFC, Body Area Network, etc.). In another example, the armband device 2800 may communicate indirectly with the monitoring device 104 and/or remote server 102, such as via the portable gateway 108. In another example, the armband device 2800 may not communicate with the monitoring device 104 and may instead only communicate with the remote server 102 (e.g., via the portable gateway).

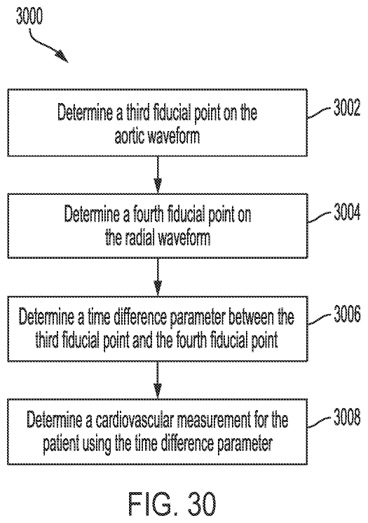

Accordingly, the monitoring device 104 and/or remote server 102 may use the radial waveform to determine a time difference parameter, such as by using the RF-based aortic region waveform in comparison with the RF-based radial waveform, and an additional cardiovascular measurement for the patient using the time difference parameter. In some implementations, the monitoring device 104 and/or remote server 102 may determine the time difference parameter and cardiovascular measurement using a process similar to the sample process 2400 discussed above. For example, FIG. 30 shows a sample process flow 3000 that can be implemented, for example, by the monitoring device 104 (e.g., via the microcontroller 606 of FIG. 6), though it should be understood that the sample process flow 3000 may be implemented via any of the embodiments of a cardiovascular monitoring unit 100 described herein or their equivalents. Moreover, the remote server 102 may alternatively or additionally implement the sample process 3000.

As shown in FIG. 30, the monitoring device 104 and/or the remote server 102 determines a third fiducial point on the aortic region waveform at step 3002. For example, the monitoring device 104 and/or remote server 102 may identify one of the fiducial points 2204-2212 of FIG. 22 as the third fiducial point for the aortic region waveform. The third fiducial point may be the same as the first fiducial point identified at step 2402 of FIG. 24, or the third fiducial point may be different from the first fiducial point identified at step 2402 of FIG. 24. The monitoring device 104 and/or remote server 102 also determines a fourth fiducial point on the radial waveform at step 3004. For example, the monitoring device 104 and/or remote server 102 may identify one of the fiducial points 2904-2914 of FIG. 29 as the fourth fiducial point for the radial waveform.

Once the monitoring device 104 and/or remote server 102 has identified the third and fourth fiducial points, the monitoring device 104 and/or remote server 102 determines a time difference parameter (e.g., a second time difference parameter compared to the first time difference parameter determined with respect to FIG. 24 above) between the third and fourth fiducial points at step 3006. In implementations, the monitoring device 104 and/or remote server 102 may determine the time difference parameter similarly to the process described above with respect to step 2406 of FIG. 24. For example, the monitoring device 104 and/or remote server 102 may identify the time difference between when the third fiducial point occurs and when the fourth fiducial point occurs, where the third fiducial point and fourth fiducial point fall on similar portions of the aortic region and radial waveforms, respectively.

After determining the time difference parameter, the monitoring device 104 and/or remote server 102 determines a cardiovascular measurement for the patient (e.g., a second cardiovascular measurement compared to the first cardiovascular measurement determined with respect to FIG. 24 above) using the time difference parameter at step 3008. In implementations, the monitoring device 104 and/or remote server 102 may determine the cardiovascular measurement similarly to the processes described above with respect to step 2408 of FIG. 24. As an illustration, the monitoring device 104 and/or remote server 102 may use time difference parameter to calculate a PTT measurement, PWV measurement, blood pressure measurement, and so on. In various implementations, the monitoring device 104 and/or remote server 102 uses the radial waveform from the armband device 2800 to determine a secondary cardiovascular measurement. For example, the monitoring device 104 and/or remote server 102 may use the secondary cardiovascular measurement to confirm the cardiovascular measurement determined from the RF-based aortic region waveform and light-based arterial waveform, discussed above.

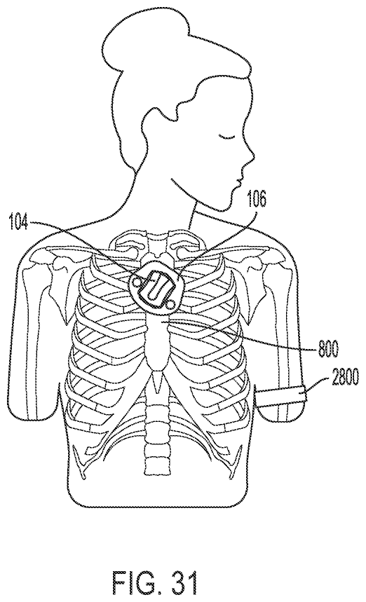

The armband device 2800 shown in FIG. 28 is an example device, and other secondary devices and/or other locations for a secondary device on the patient may be used. As an example, FIG. 31 illustrates the armband device 2800 positioned over the patient's upper arm (e.g., above the brachial artery). The armband device may therefore provide RF signals including information about an RF-based brachial waveform, which may also be shaped similarly to the aortic region waveform.

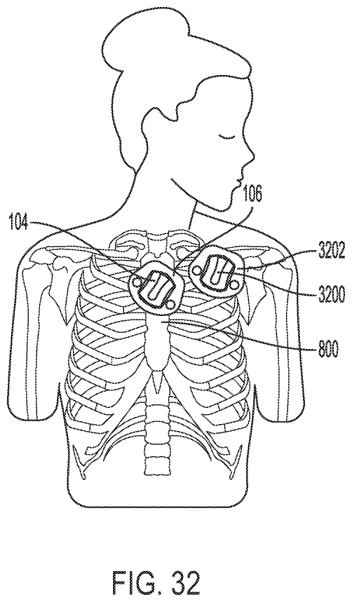

As an example, FIG. 32 illustrates the cardiovascular monitoring unit 100 including the monitoring device 104 mounted on the adhesive patch 106 over the patient's sternum 800 along with a secondary monitoring device 3200. The secondary monitoring device 3200 in the embodiment of FIG. 32 is configured similarly to the monitoring device 104 (e.g., including a structure similar to the structure of the monitoring device 104 discussed above with respect to FIGS. 6 and 7 but without the at least one light source and light sensor). Additionally, the secondary monitoring device 3200 is also mounted on a secondary adhesive patch 3202, which may be configured the same as the adhesive patch 106 or may be configured differently from the adhesive patch 106 (e.g., without ECG electrodes). As shown in FIG. 32, the secondary monitoring device 3200 mounted on the secondary adhesive patch 3202 near the patient's clavicle (e.g., over the subclavian artery). The secondary monitoring device 104 may thus provide RF signals including information about an RF-based subclavian waveform, which may also be shaped similarly to the aortic region waveform.

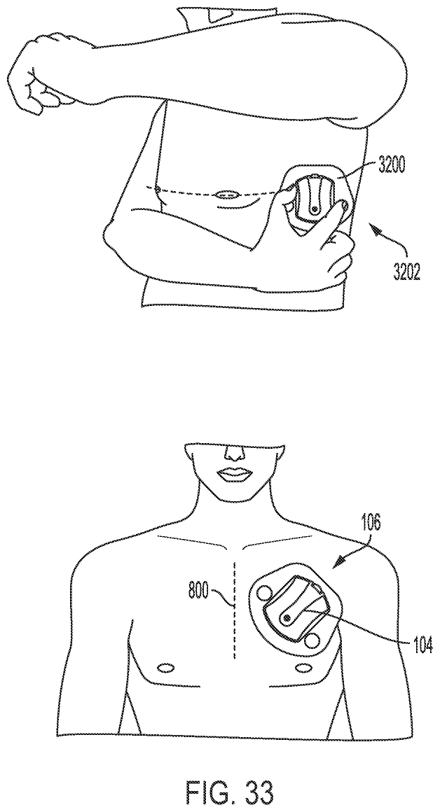

As another example, FIG. 33 illustrates the cardiovascular monitoring unit 100 including the monitoring device 104 mounted on the adhesive patch 106 over and/or near the patient's sternum 800, as well as the secondary monitoring device 3200 mounted on the secondary adhesive patch 3202 on the patient's side. The secondary adhesive patch 3202 may be mounted so as to provide additional RF signals including information about the aortic region waveform, so as to provide RF signals including information about a waveform of the superior mesenteric artery of the patient, or RF signals including information about other arteries in the patient's torso.

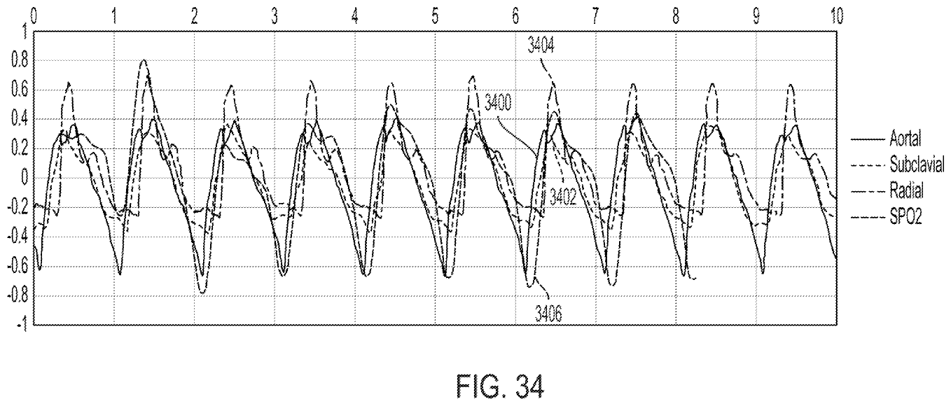

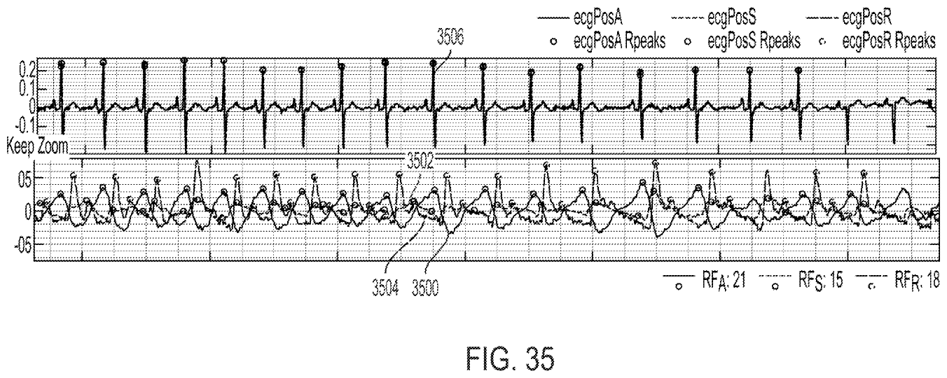

The monitoring device 104 and/or remote server 102 may use RF signals provided by other types of secondary devices in a process similar to the example process 3000 of FIG. 30 to determine additional and/or secondary cardiovascular measurements for the patient. In some implementations, the monitoring device 104 and/or remote server 102 may use the RF signals provided by other types of secondary devices in combination with other signals discussed above, such as the light-based sensor signals including information about the light-based arterial waveform and/or the ECG signals, to determine additional and/or secondary cardiovascular measurements. For example, FIG. 34 illustrates a graph showing an RF-based arterial waveform 3400, an RF-based subclavian waveform 3402, an RF-based radial waveform 3404, and a light-based arterial waveform 3406 on the same timeline (in seconds). In some implementations, the monitoring device 104 and/or remote server 102 may use corresponding fiducial points between any two of the waveforms 3400, 3402, 3404, and 3406 to determine a cardiovascular measurement. As another example, FIG. 35 illustrates a graph showing an RF-based arterial waveform 3500, an RF-based subclavian waveform 3502, an RF-based radial waveform 3504, and an ECG waveform 3506 on the same timeline (in seconds). In some implementations, the monitoring device 104 and/or remote server 102 may similarly use corresponding points between any two of the waveforms 3500, 3502, 3504, and 3506 to determine a cardiovascular measurement.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1. A medical monitoring system for remote monitoring of radiofrequency (RF)-based and light-based physiological information of a patient, comprising:
an RF transmitter configured to generate RF waves, wherein the RF transmitter is configured to be placed on a first location of the patient such that the generated RF waves are directed towards an aortic region of the patient comprising at least one of an aorta or one or more branching arteries proximate to the aorta;
an RF receiver and associated RF circuitry configured to receive RF waves reflected from the aortic region of the patient, wherein the RF circuitry is configured to provide RF sensor signals, based on the received RF waves, comprising information about an RF-based aortic region waveform of the patient;
at least one light source configured to generate light of one or more predetermined frequencies, wherein the at least one light source is configured to be placed on the first location of the patient such that the generated light is directed towards one or more arteries below skin on a thorax of the patient;
a light sensor and associated light sensor circuitry configured to receive light reflected from the one or more arteries below the skin, wherein the light sensor circuitry is configured to provide light sensor signals, based on the received light, comprising information about a light-based arterial waveform of the patient;
a memory implemented in a non-transitory media; and
a processor in communication with the memory; the processor configured to
determine a first fiducial point on the RF-based aortic region waveform;
determine a second fiducial point on the light-based arterial waveform;
determine a time difference parameter between the first fiducial point and the second fiducial point; and
determine, using the time difference parameter and a distance along an arterial tree between the aortic region and the one or more arteries below the skin, a pulse wave velocity of the patient.

2. The medical monitoring system of claim 1, wherein the first location comprises a location on skin above a sternum of the patient.

3. The medical monitoring system of claim 1, further comprising
a second RF transmitter configured to generate a second set of RF waves, wherein the second RF transmitter is configured to be placed on a second location of the patient such that the second set of RF waves are directed towards an artery of the patient at the second location; and
a second RF receiver and associated second RF circuitry configured to receive a second set of RF waves reflected from the artery at the second location of the patient, wherein the second RF circuitry is configured to provide a second set of RF signals, based on the received second set of RF waves, comprising information about an RF-based waveform of the artery at the second location.

4. The medical monitoring system of claim 3, wherein the processor is further configured to
determine a third fiducial point on the RF-based aortic region waveform;
determine a fourth fiducial point on the RF-based waveform of the artery at the second location; and
determine a second time difference parameter between the third fiducial point and the fourth fiducial point.

5. The medical monitoring system of claim 4, wherein the processor is further configured to determine, using the second time difference parameter and a distance along the arterial tree between the aortic region and the artery at the second location, a second pulse wave velocity of the patient.

6. The medical monitoring system of claim 4, wherein the processor is further configured to determine, using at least one of the second pulse wave velocity or the second time difference parameter, a blood pressure of the patient.

7. The medical monitoring system of claim 3, wherein the second location comprises a location above a radial artery of the patient, and wherein the RF-based waveform of the artery at the second location comprises an RF-based radial waveform of the patient.

8. The medical monitoring system of claim 3, wherein the second location comprises a location above a subclavian artery of the patient, and wherein the RF-based waveform of the artery at the second location comprises an RF-based subclavian waveform of the patient.

9. The medical monitoring system of claim 3, wherein the second location comprises a location above a brachial artery of the patient, and wherein the RF-based waveform of the artery at the second location comprises an RF-based brachial waveform of the patient.

10-22. (canceled)

23. The medical monitoring system of claim 1, wherein the processor is further configured to determine, using at least one of the pulse wave velocity or the time difference parameter, a blood pressure of the patient.

24-29. (canceled)

30. The medical monitoring system of claim 23, wherein the processor is configured to determine the blood pressure of the patient based on a predetermined function of a logarithm of a square of the pulse wave velocity.

31. (canceled)

32. (canceled)

33. The medical monitoring system of claim 1, wherein the time difference parameter between the first fiducial point and the second fiducial point is one of a plurality of time difference parameters between fiducial points of the RF-based aortic region waveform and light-based arterial waveform over a summary time period.

34. The medical monitoring system of claim 33, wherein the processor is further configured to determine the plurality of time difference parameters by
determining a plurality of first fiducial points on the RF-based aortic region waveform;
determining a plurality of second fiducial points on the light-based arterial waveform; and
determining a time difference parameter between each first fiducial point and corresponding second fiducial point.

35. (canceled)

36. (canceled)

37. The medical monitoring system of claim 33, wherein the processor is further configured to determine, using the plurality of time difference parameters, a summary time difference parameter for the summary time period.

38. (canceled)

39. The medical monitoring system of claim 33, wherein the processor is further configured to determine, using the plurality of time difference parameters and the distance along the arterial tree between the aortic region and the one or more arteries below the skin, a summary pulse wave velocity of the patient for the summary time period.

40. (canceled)

41. The medical monitoring system of claim 1, further comprising a patch configured to be adhesively attached to the first location of the patient.

42. The medical monitoring system of claim 41, wherein the RF transmitter and the RF receiver and associated RF circuitry are configured to be mounted onto the patch.

43-46. (canceled)

47. The medical monitoring system of claim 1, further comprising two or more ECG electrodes, wherein the processor is further configured to receive ECG signals from the two or more ECG electrodes.

48. The medical monitoring system of claim 1, further comprising a monitoring device, wherein the monitoring device comprises the memory, the processor, and at least some of the RF transmitter, the RF receiver and associated RF circuitry, the at least one light source, or the light sensor and associated light sensor circuitry.

49. The medical monitoring system of claim 1, further comprising a remote server, wherein the remote server comprises the memory and the processor.

50-152. (canceled)
